(19) United States
(12) Patent Application Publication
(10) Pub. No.: US 2012/0282255 A1
(43) Pub. Date: Nov. 8, 2012

(54) METHODS AND COMPOSITIONS FOR THE TREATMENT OF ALCOHOLISM AND ALCOHOL DEPENDENCE

(76) Inventor: Greg Plucinski, Lexington, KY (US)

(21) Appl. No.: 13/441,337

(22) Filed: Apr. 6, 2012

Related U.S. Application Data
(60) Provisional application No. 61/472,947, filed on Apr. 7, 2011.

(51) Int. Cl. A61K 38/26; A61K 31/4985; A61K 31/40; A61K 31/403; A61K 31/522; A61K 31/4025; A61K 31/519; A61K 31/4375; A61P 25/08; A61P 25/30; A61K 38/08; A61K 38/02; A61K 31/485; A61K 31/55; A61K 31/135; A61K 31/5415; A61K 31/167; A61K 31/551; A61K 31/451; A61K 31/15; A61K 31/439; A61K 31/517; A61K 31/35; A61P 25/34; A61P 25/32; A61P 3/04; A61P 25/36; A61P 25/22; A61P 25/18; A61P 25/28; A61K 39/395

(52) U.S. Cl. 424/134.1; 514/11.7; 514/249; 514/423; 514/412; 514/263.21; 514/422; 514/264.1; 514/274; 514/280; 514/21.6; 514/1.1; 514/282; 514/279; 514/289; 514/217; 514/646; 514/226.2; 514/617; 514/220; 514/325; 514/645; 514/299; 514/266.2; 514/259.2; 514/455

(57) ABSTRACT

The present invention provides for compositions and methods for treating or preventing addictive and compulsive diseases and disorders, particular alcohol-related diseases and disorders, disclosed herein. The GLP activators of the present invention are effective against various alcohol and drug dependency diseases. In accordance with the invention, the present compositions and methods can be used to intercede upstream or downstream in the signal transduction cascade involved in GLP action to treat various alcohol and drug dependency diseases. In one embodiment, the synthesis or release of endogenous GLP can be stimulated. In another embodiment, the endogenous synthesis or release of another molecule active in the cascade downstream from GLP, (e.g., a molecule produced in response to GLP binding to a receptor), can be stimulated. Accordingly, the methods and compositions of the invention are useful for preventing, treating, diagnosing, or monitoring the progression various alcohol and drug dependency diseases disclosed herein.

### CROSS REFERENCE TO RELATED APPLICATIONS

The present application hereby claims the benefit of the provisional patent application of the same title, Ser. No. 61/472,947, filed on Apr. 7, 2011, the disclosure of which is hereby incorporated by reference in its entirety.

### BACKGROUND

Alcoholism may be viewed as a disease, a drug addiction, a learned response to crisis, a symptom of an underlying psychological or physical disorder, or a combination of these factors. Most approaches to the treatment of alcoholism require the alcoholic person to recognize his/her illness and to abstain from alcohol. Treatment programs then vary according to the accepted definition and theory of cause of alcoholism. Treatment types include combinations of: psychological rehabilitative treatments; organized self-help groups; aversion therapy based on behavior modification; injections of vitamins or hormones, and the use of abstinence-maintaining drugs. The present invention relates to the latter type of treatments.

One of the drug treatments of alcoholism, initiated in 1948 by Eric Jacobsen of Denmark, uses disulfuram (tetraethylthiuram disulfide). The presence of disulfuram in the drinker's body causes a reaction of hot flushing, nausea, vomiting, a sudden sharp drop of blood pressure, pounding of the heart, and even a feeling of impending death. These symptoms, usually known as “acetaldehyde syndrome” or “efecto adverso tipo-disulfiiram”, result from an accumulation of the highly toxic first product of alcohol metabolism, acetaldehyde.

However, following disulfuram treatment cases of respiratory depression, cardiovascular collapse, cardiac arrhythmia, myocardium infarct, and sudden or unexpected death have occurred.

Many anti-alcoholism agents have been proposed, including the following: opioid antagonists, such as naltrexone, naloxone and nalmefene (cf. U.S. Pat. Nos. 4,882,335 and 5,086,058); acyl L-carnitine gamma-hydroxybutyrates (cf. EP 616,805-A1), gamma-hydroxybutyric acid salts (cf. U.S. Pat. No. 4,983,632) and gamma-hydroxybutyric acid amides (cf. WO 9806690-A1); 2-pyrimidinyl-1-piperazine derivatives such as ipsapirone (cf. U.S. Pat. No. 4,895,848); pyrrolidine derivatives (cf. U.S. Pat. No. 5,935,980); cholinesterase inhibitor, such as galanthamine (cf. U.S. Pat. No. 5,932,238); serotonin reuptake inhibitors, such as fluoxetine, and the combination of the later with opioid antagonists (cf. WO 9609047-A1).

GLP-1 (Glucagon-Like Peptide-1) is known as an incretin hormone which is secreted from digestive tracts upon ingestion of food to act on the pancreas and stimulate insulin secretion. As a hormone exhibiting a similar action, there is GIP (Gastric Inhibitory Polypeptide or Glucose-dependent Insulinotropic Polypeptide). This incretin effect is suggested to be absent or reduced in patients with type 2 diabetes, compared with healthy persons, and this is considered as one of causes high blood glucose. For example, it is reported that in patients with type 2 diabetes, blood GLP-1 level is lowered, while blood GIP level is normal. As a result of administering the incretin hormones to patients with type 2 diabetes, there is no difference upon the insulin secretion-stimulating activity of GLP-1 between the patients and healthy persons, while the insulin secretion-stimulating activity of GIP is significantly lower in the patients than healthy persons. Accordingly, the response of the patients with diabetes to GLP-1 is maintained; thus, a GLP-1 preparation compensating for its shortage can be expected to serve as a medicine for treatment of diabetes.

The action of GLP-1 on insulin secretion is characterized by glucose level dependent that GLP-1 does not stimulate insulin secretion in the blood glucose level of 110 mg/dL or less. That is, Administration of GLP-1 has clinical advantages that lower possibility of hypoglycemia, and suppress the excessive insulin secretion so that the exhaustion of the pancreas is prevented. While a sulfonylurea, used mainly in treatment of type 2 diabetes, closes ATP-sensitive K+ channels continuously to promote insulin secretion it causes low blood glucose, exhaustion of the pancreas by excessive stimulation of β cells, and secondary failure in administration for a long period of time. Accordingly, the pharmacological characteristics of GLP-1 are very useful and different from those of the conventional medicine for diabetes.

GLP-1 also has the following characteristics: suppression of glucagon secretion, delay of gastric emptying, suppression of stomach acid secretin, action on the brain to suppress appetite, promotion of insulin synthesis in pancreatic β cells and proliferation of pancreatic β cells. Therefore, GLP-1 is considered not only effective for treatment of diabetes by antagonizing the cause of high blood glucose such as hyperglucagonemia in type 2 diabetes, but also effective for treatment of obesity.

However, as GLP-1 is the polypeptide made up of 30 or 31 amino acids, it is digested upon oral ingestion and decomposed by digestive enzyme in the digestive tract, and is thus not absorbed. The administration thereof by intravenous injection or subcutaneous injection of GLP-1 is attempted at present. Further, it is known that GLP-1 undergoes decomposition with dipeptidyl peptidase IV (DPPIV) present in blood or tissues so that the half-life thereof in the living body is as very short as 1 to 2 minutes, thus giving rise to an obstacle to clinical applications.

### BRIEF SUMMARY

The present invention relates generally to compositions and methods for the treatment of alcoholism. More specifically, the present invention relates to the use of human glucagon-like peptide-1 (GLP-1), GLP-1 receptor agonist or derivatives and mixtures thereof absorbed via a mucous membrane in the oral cavity, lung, nose or intestines, production thereof and a method of using the same.

In one embodiment, the present invention provides compositions and methods for treating or preventing an alcohol-related disease or disorder comprising administering to a subject a therapeutically effective amount of glucagon-like peptide-1 (GLP-1), GLP-1 receptor agonist or derivatives and mixtures thereof. In another embodiment, the present invention provides compositions and methods for treating or preventing an alcohol-related disease or disorder comprising administering to a subject a therapeutically effective amount of glucagon-like peptide-1 (GLP-1), GLP-1 receptor agonist or derivatives and mixtures thereof and at least one anti-alcohol agent or compound, and optionally other therapeutic agents. The present invention further encompasses the adjunctive use of psychosocial management techniques.

In one embodiment, the present invention provides methods for treating or preventing an alcohol-related disease or disorder in a subject comprising administering an effective amount of glucagon-like peptide-1 (GLP-1) and at least one compound, and analog, homolog, derivative, modification, and pharmaceutically acceptable salt thereof, selected from the group consisting of serotonergic agents, serotonin antagonists, selective serotonin re-uptake inhibitors, serotonin receptor antagonists, opioid antagonists, dopaminergic agents, dopamine release inhibitors, dopamine antagonists, norepinephrine antagonists, GABA agonists, GABA inhibitors, GABA receptor antagonists, GABA channel antagonists, glutamate agonists, glutamate antagonists, glutamine agonists, glutamine antagonists, anti-convulsant agents, NMDA-blocking agents, calcium channel antagonists, carbonic anhydrase inhibitors, neurokinins, small molecules, peptides, vitamins, co-factors, anti-orexin agents, regulators of cannabanoid receptor-1, and Corticosteroid Releasing Factor antagonists. In one aspect, the neurokinin is NPY. The present invention further encompasses administering other small molecules and peptides.

In one embodiment, the present invention provides methods for treating or preventing an alcohol-related disease or disorder in a subject comprising administering an effective amount of glucagon-like peptide-1 (GLP-1) and at least one serotonin receptor antagonist selected from the group consisting of 1-(−)-cocaine, 2-bromo-CSD (BOL), 3-tropanyl-indole-3-carboxylate, 3-tropanyl-indole-3-carboxylate methiodide, amitriptine, carpipramine, chlorpromazine, cinanserin, clocapramine, clozapine, cyproheptadine, fluvoxamine, granisetron, imipramine, ketanserin, levomepromazine, LSD, LY-278,584, LY-53,857, MDL100907, MDL-11939, metergoline, methiothepin, methysergide, mianserin, milnacipran, mirtazapine, mosapramine, NAN-190, nortriptyne, olanzapine, paroxetine, perospirone, piperazine, p-NPPL, quetiapine, risperidone, ritanserin, sarpogrelate, SB-206553, SDZ-205,557, trazodone and xylamidine. In the preferred embodiment, active agents are exerted only in the peripheral nervous system. In one embodiment, the peripheral serotonin receptor antagonist is administered in an amount of at least about 0.01 mg per 100 kg body weight, preferably at least about 0.1 mg per 100 kg body weight, and more preferably at least about 1 mg per 100 kg body weight. The peripheral serotonin receptor antagonist is generally administered in an amount of at most about 500 mg per 100 kg body weight, preferably at most about 250 mg per 100 kg body weight, and more preferably at most about 100 mg per 100 kg body weight.

In one embodiment, the alcohol-related disease or disorder being treated includes, but is not limited to, early-onset alcoholic, late-onset alcoholic, alcohol-induced psychotic disorder with delusions, alcohol abuse, excessive drinking, heavy drinking, problem drinking, alcohol intoxication, alcohol withdrawal, alcohol intoxication delirium, alcohol withdrawal delirium, alcohol-induced persisting dementia, alcohol-induced persisting amnestic disorder, alcohol dependence, alcohol-induced psychotic disorder with hallucinations, alcohol-induced mood disorder, alcohol-induced or associated bipolar disorder, alcohol-induced or associated posttraumatic stress disorder, alcohol-induced anxiety disorder, alcohol-induced sexual dysfunction, alcohol-induced sleep disorder, alcohol-induced or associated gambling disorder, alcohol-induced or associated sexual disorder, alcohol-related disorder not otherwise specified, alcohol intoxication, and alcohol withdrawal.

In one embodiment, the present invention provides compositions and methods for reducing the frequency of alcohol consumption compared with the frequency of alcohol consumption before the treatment. One of ordinary skill in the art will appreciate that the frequency can be compared with prior consumption by the subject or with consumption by a control subject not receiving the treatment. In one aspect, the type of alcohol consumption is heavy drinking. In another aspect, it is excessive drinking

In one embodiment, the present invention provides compositions and methods for reducing the quantity of alcohol consumed in a subject compared with the amount of alcohol consumed before the treatment or compared with the alcohol consumption by a control subject not receiving the treatment.

One of ordinary skill in the art will appreciate that in some instances a subject being treated for and addictive disorder is not necessarily dependent. Such subjects include, for example, subjects who abuse alcohol, drink heavily, drink excessively, are problem drinkers, or are heavy drug users. The present invention provides compositions and methods for treating or preventing these behaviors in non-dependent subjects.

In one embodiment of the invention, the present invention provides compositions and methods for improving the physical or psychological sequelae associated with alcohol consumption compared with a control subject not receiving the treatment.

In one embodiment, the present invention provides compositions and methods for increasing the abstinence rate of a subject compared with a control subject not receiving the treatment.

In one embodiment, the present invention provides compositions and methods for reducing the average level of alcohol consumption in a subject compared with the level of alcohol consumption before the treatment or compared with the level of alcohol consumption by a control subject not receiving the treatment.

In one embodiment, the present invention provides compositions and methods for reducing alcohol consumption and for increasing abstinence compared with the alcohol consumption by the subject before treatment or with a control subject not receiving the treatment.

In one embodiment, the present invention provides compositions and methods for treating a subject with a predisposition to early-onset alcoholism.

In one embodiment, the present invention provides compositions and methods for treating a subject with a predisposition to late-onset alcoholism.

One of ordinary skill in the art will appreciate that there are multiple parameters or characteristics of alcohol consumption which may characterize a subject afflicted with an alcohol-related disease or disorder. It will also be appreciated that combination therapies may be effective in treating more than one parameter, and that there are multiple ways to analyze the effectiveness of treatment. The parameters analyzed when measuring alcohol consumption or frequency of alcohol consumption include, but are not limited to, heavy drinking days, number of heavy drinking days, average drinking days, number of drinks per day, days of abstinence, number of individuals not drinking heavily or abstinent over a given time period, and craving. Both subjective and objective measures can be used to analyze the effectiveness of treatment. For example, a subject can self-report according to guidelines and procedures established for such reporting. The procedures can be performed at various times before, during, and after treatment. Additionally, assays are available for measuring alcohol consumption. These assays include breath alcohol meter readings, measuring serum CDT and GGT levels, and measuring 5-HTOL urine levels.

The present invention further provides adjunctive therapies to be used in conjunction with the combination drug therapies. The present invention further provides adjunctive therapy or treatment wherein the subject is also submitted to a psychosocial management program. Psychosocial management programs are known in the art and include, but are not limited to, Brief Behavioral Compliance Enhancement Treatment, Cognitive Behavioral Coping Skills Therapy, Motivational Enhancement Therapy, Twelve-Step Facilitation Therapy (Alcoholics Anonymous), Combined Behavioral Intervention, Medical Management, psychoanalysis, psychodynamic treatment, and Biopsychosocial, Report, Empathy, Needs, Advice, Direct Advice and Assessment. The present invention further encompasses the use of additional adjunct therapies and treatment, including hypnosis and acupuncture.

In one embodiment, at least one of the additional compounds is a serotonin receptor antagonist. In one aspect, the serotonin receptor is the serotonin-3 receptor. In one aspect, the compound is ondansetron. In one embodiment, at least three different compounds are administered to the subject.

It will be appreciated by one of ordinary skill in the art that the two or more compounds being administered do not necessarily have to be administered at the same time or in equal doses. In one aspect, the compounds being administered as part of the drug combination therapy are separately administered. In another aspect, a first compound is administered before a second compound is administered. In yet another aspect, a first compound and a second compound are administered nearly simultaneously. In a further aspect, the first compound is administered subsequent to administration of the second compound.

The invention further provides pharmaceutical compositions comprising compounds of the invention. The pharmaceutical composition may comprise one or more compounds of the invention, and biologically active analogs, homologs, derivatives, modifications, and pharmaceutically acceptable salts thereof, and a pharmaceutically acceptable carrier. In one embodiment, the compounds are administered as a pharmaceutical composition.

The route of administration can vary depending on the type of compound being administered. In one aspect, the compounds are administered via routes such as oral, topical, rectal, intramuscular, intramucosal, intranasal, inhalation, ophthalmic, and intravenous.

The present invention further provides for administration of a compound of the invention as a controlled-release formulation.

In one embodiment, the present invention provides administering at least two compounds where the compounds are glucagon-like peptide-1 (GLP-1) and at least one compound selected from the group consisting of topiramate, ondansetron, and naltrexone. In one aspect, two compounds are administered in addition to GLP-1.

In one embodiment, the present invention provides compositions and methods for treating alcohol-related diseases and disorders using pharmaceutical compositions comprising effective amounts of GLP-1 in combination with topiramate and ondansetron.

The dosage of the active compound(s) being administered will depend on the condition being treated, the particular compound, and other clinical factors such as age, sex, weight, and health of the subject being treated, the route of administration of the compound(s), and the type of composition being administered (tablet, gel cap, capsule, solution, suspension, inhaler, aerosol, elixir, lozenge, injection, patch, ointment, cream, etc.). It is to be understood that the present invention has application for both human and veterinary use.

For example, in one embodiment relating to oral administration to humans, a dosage of between approximately 0.1 and 300 mg/kg/day, or between approximately 0.5 and 50 mg/kg/day, or between approximately 1 and 10 mg/kg/day, is generally sufficient, but will vary depending on such things as the disorder being treated, the length of treatment, the age, sex, weight, and/or health of the subject, etc. The drugs can be administered in formulations that contain all drugs being used, or the drugs can be administered separately. In some cases, it is anticipated that multiple doses/times of administration will be required or useful. Additionally, for some treatment regimens, at least two compounds will be used. The present invention further provides for varying the length of time of treatment.

Topiramate is disclosed herein as a drug useful in combination drug therapy. In one embodiment, topiramate is provided at a dosage ranging from about 15 mg/day to about 2500 mg/day. In one aspect, topiramate is administered at a dosage ranging from about 25 mg/day to about 1000 mg/day. In yet another aspect, topiramate is administered at a dosage ranging from about 50 mg/day to about 500 mg/day. In one aspect, topiramate is administered at a dosage of about 400 mg/day. In another aspect, topiramate is administered at a dosage of 400 mg/day. In a further aspect, topiramate is administered at a dosage of about 300 mg/day. In yet a further aspect, topiramate is administered at a dosage of about 275 mg/day. In one aspect, topiramate is administered at a dose of about 1 mg/day.

In one embodiment, topiramate is provided at a dose of about 1 mg/kg. In one aspect, topiramate is provided at a dose of about 10 mg/kg. In one aspect, topiramate is provided at a dose of about 100 mg/kg. In one embodiment, topiramate is administered at a dosage ranging from about 0.1 mg/kg/day to about 100 mg/kg/day.

The present invention further provides for the use of other drugs such as naltrexone as part of the drug combination therapy disclosed herein. In one embodiment, naltrexone is administered at a dose of about 10 mg/day. In one aspect, naltrexone is administered at a dosage at a dosage of about 50 mg/day. In one aspect, naltrexone is administered at a dosage of about 100 mg/day. In one aspect, naltrexone is administered at a dosage ranging from about 1 mg to about 300 mg per application. In another aspect, naltrexone is administered at a dosage ranging from about 10 mg to about 50 mg per application. In a further aspect of the invention, naltrexone is administered at a dosage of about 25 mg per application. In one embodiment, naltrexone is administered at least once a month. In a further embodiment, naltrexone is administered once a month. In one embodiment, naltrexone is administered at least once a week. In another embodiment, naltrexone is administered at least once a day. In a further embodiment, naltrexone is administered at least twice a day. In one aspect, naltrexone is administered twice a day.

In one embodiment where at least two compounds are administered along with GLP-1, topiramate and naltrexone are administered. In one aspect, topiramate is administered at a dosage of as much as about 400 mg/day and naltrexone is administered at a dosage of about 25 mg/application to about 150 mg/application. In a further aspect, topiramate is administered at a dosage of about 300 mg/day and naltrexone is administered at a dosage of about 25-50 mg/application.

Ondansetron is disclosed herein as a drug useful in the combination drug therapy of the invention. The dosage and treatment regimen for administering ondansetron when it is being used as one compound of a combination therapy can be varied based on the other drug or drugs with which it is being administered, or based on other criteria such as the age, sex, health, and weight of the subject. The present invention therefore provides for the use of ondansetron at varying doses such as about 0.01 μg/kg, about 0.1 μg/kg, about 1.0 μg/kg, about 5.0 μg/kg, about 10.0 μg/kg, about 0.1 mg/kg, about 1.0 mg/kg, about 5.0 mg/kg, and about 10.0 mg/kg. In another embodiment, ondansetron is administered at a dosage ranging from about 0.01 μg/kg to about 100 μg/kg per application. In one aspect, ondansetron is administered at a dosage ranging from about 0.1 μg/kg to about 10.0 μg/kg per application. In yet another aspect, ondansetron is administered at a dosage ranging from about 1.0 μg/kg to about 5.0 μg/kg per application. In a further aspect, ondansetron is administered at a dosage of about 4.0 μg/kg per application. In another aspect, ondansetron is administered at a dosage of about 3.0 μg/kg per application.

In one embodiment, the results of treating a subject with a combination of two or more compounds are additive compared with the effects of using any of the compounds alone. In one aspect, the effects seen when using two or more compounds are greater than when using any of the compounds alone.

In one embodiment, the results of treating a subject with a combination of two or more compounds are synergistic compared with the effects of using the compounds alone.

In one embodiment, other compounds may be used in combination with GLP-1 and topiramate, for example, ondansetron and/or and naltrexone.

In addition to the combination treatment of at least two drugs described above, the present invention further provides for the administration of GLP-1 and at least one additional compound to treat or prevent diseases and disorders of the invention, including, but not limited to, disulfuram, acamprosate, sertraline, galanthamine, nalmefene, naloxone, desoxypeganine, benzodiazepines, neuroleptics, risperidone, rimonabant, trazodone, baclofen, regulators of cannabanoid receptor-1, regulators of orexin, and aripiprazole. One of ordinary skill in the art will appreciate that in some cases the combination therapy using these additional compounds will have additive effects and in some cases synergistic effects. Methods for testing these combinations and analyzing the results are known in the art.

In addition to the combination drug therapy described herein for treating or preventing addiction-related diseases and disorders such as alcohol-related diseases and disorders, additional types of compounds can be administered to treat further the addiction-related diseases and disorders or to treat other diseases and disorders. The additional types of compounds include, but are not limited to, adrenergics, adrenocortical steroids, adrenocortical suppressants, aldosterone antagonists, amino acids, analeptics, analgesics, anorectic compounds, anorexics, anti-anxiety agents, antidepressants, antihypertensives, anti-inflammatories, antinauseants, antineutropenics, antiobsessional agents, antiparkinsonians, antipsychotics, appetite suppressants, blood glucose regulators, carbonic anhydrase inhibitors, cardiotonics, cardiovascular agents, choleretics, cholinergics, cholinergic agonists, cholinesterase deactivators, cognition adjuvants, cognition enhancers, hormones, memory adjuvants, mental performance enhancers, mood regulators, neuroleptics, neuroprotectives, psychotropics, relaxants, sedative-hypnotics, stimulants, thyroid hormones, thyroid inhibitors, thyromimetics, cerebral ischemia agents, vasoconstrictors, and vasodilators.

The present invention further encompasses biologically active analogs, homologs, derivatives, and modifications of the compounds of the invention. Methods for the preparation of such compounds are known in the art. In one aspect, the compounds are topiramate, naltrexone, and ondansetron.

The compositions and methods described herein for treating or preventing alcohol-related diseases and disorders are also useful for treating or preventing other addiction-related diseases and disorders and impulse control disorders. In one aspect, the compositions and methods elicit an indirect effect on CMDA neurons. Such effects may be elicited, for example, by regulating serotonergic, opiate, glutamate, or γ-amino-butyric acid receptors. In one aspect, the addictive diseases and disorders include eating disorders, impulse control disorders, nicotine-related disorders, methamphetamine-related disorders amphetamine-related disorders, cannabis-related disorders, cocaine-related disorders, hallucinogen use disorders, inhalant-related disorders, benzodiazepine abuse or dependence related disorders, and opioid-related disorders.

The compositions and methods described herein are also useful for treating or preventing heavy drug use, including, but not limited to, cocaine, methamphetamine, other stimulants, phencyclidine, other hallucinogens, marijuana, sedatives, tranquilizers, hypnotics, and opiates. It will be appreciated by one of ordinary skill in the art that heavy use or abuse of a substance does not necessarily mean the subject is dependent on the substance.

The compositions and methods of the present invention are also useful as a multi-faceted therapy approach to treating and regulating weight loss, obesity, and weight gain. Therefore, the therapy of the present invention for the treatment of addictive disorders and associated impulsivity, including obesity, is a new and useful therapy. Based on the data and descriptions provided herein, as well as what is known in the art, one of ordinary skill in the art will know how to combine and use drugs such as GLP-1, topiramate, ondansetron, and naltrexone in multiple formats to optimize the invention. These pharmacological formats include (but are not limited to) tablets, gel caps, capsules, chewable and orally absorbable materials (for example, sublingual tablets), elixirs, suspensions, inhalants, sprays, patches, ointments and balms, long-acting intramuscular injections (with FDA-approved polylactide capsules or nanotechnology), and intravenous, subcutaneous, intramucosal, or any other avenues for injection.

In one embodiment, the present invention provides compositions and methods for treating obesity or being overweight comprising administering to a subject in need thereof an effective amount of GLP-1 and at least one compound, or analog, derivative, modification, or pharmaceutically acceptable salt thereof, selected from the group consisting of serotonergic agents, serotonin antagonists, selective serotonin re-uptake inhibitors, serotonin receptor antagonists, opioid antagonists, dopaminergic agents, dopamine release inhibitors, dopamine antagonists, γ-amino-butyric acid agonists, γ-amino-butyric acid inhibitors, γ-amino-butyric acid receptor antagonists, γ-amino-butyric acid channel antagonists, glutamate agonists, glutamate antagonists, anti-convulsant agents, and NMDA-blocking agents, thereby treating or preventing, optionally in combination with at least one additional therapeutically active compound.

In one embodiment of treating obesity, the additional therapeutically active compound is selected from the group consisting of antidiabetic agents, antihyperlipidemic agents, antiobesity agents, antihypertensive agents, and agents for the treatment of complications resulting from or associated with diabetes.

In one embodiment, the compositions and methods of the present invention are also useful for treating or preventing an addiction-related disease or disorder other than alcohol-related diseases and disorders and weight control diseases and disorders. The method comprises administering an effective amount of at least two compounds of the invention, and analogs, derivatives, modifications, or pharmaceutically acceptable salts thereof. In one aspect, the compounds include, but are not limited to, serotonergic agents, serotonin antagonists, selective serotonin re-uptake inhibitors, serotonin receptor antagonists, opioid antagonists, dopaminergic agents, dopamine release inhibitors, dopamine antagonists, norepinephrine antagonists, γ-amino-butyric acid agonists, γ-amino-butyric acid inhibitors, γ-amino-butyric acid receptor antagonists, γ-amino-butyric acid channel antagonists, glutamate agonists, glutamate antagonists, glutamine agonists, glutamine antagonists, anti-convulsant agents, N-methyl-D-aspartate-blocking agents, calcium channel antagonists, carbonic anhydrase inhibitors, neurokinins, and Corticosteroid Releasing Factor antagonists. In one aspect, the compounds are topiramate, ondansetron, and naltrexone.

The invention provides all possible combination and permutations for the use of such drugs to treat addictive diseases and disorders, either singly or in any combination. In one embodiment, the addictive disorders include, but are not limited to, eating disorders, impulse control disorders, gambling disorders, sexual disorders, nicotine-related disorders, amphetamine-related disorders, cannabis-related disorders, cocaine-related disorders, hallucinogen use disorders, inhalant-related disorders, benzodiazepine abuse- or dependence-related disorders, and opioid-related disorders. Food and eating disorders include, for example, binge eating. In one aspect, the combination pharmacotherapy is provided in conjunction with behavioral modification therapy or intervention.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### DETAILED DESCRIPTION

It must be noted that as used herein and in the appended claims, the singular forms “a”, “and”, and “the” include plural referents unless the context clearly dictates otherwise.

Unless defined otherwise, all technical and scientific terms used herein have the same meanings as commonly understood by one of ordinary skill in the art to which this invention belongs. All references, publications, patents, patent applications, and commercial materials mentioned herein are incorporated herein by reference in their entirety for all purposes including for describing and disclosing the compositions, cell lines, vectors, and methodologies which are reported in the publications which might be used in connection with the invention. Nothing herein is to be construed as an admission that the invention is not entitled to antedate such disclosure by virtue of prior invention.

In order to provide a clear and consistent understanding of the specification and claims, including the scope to be given such terms, the following definitions are provided:

As used herein, the phrase “GLP” refers to GLP-1 or GLP-2.

As used herein, the phrase “GLP molecules” refers to GLP peptides, fragments of GLP peptides, nucleic acids that encode GLP peptides or fragments, or variants thereof.

As used herein, the term “variant” or “variants” refers to variations of the nucleic acid or amino acid sequence of GLP molecules. Homologues and analogs of a GLP molecule of the invention are contemplated. Encompassed within the term “variant(s)” are nucleotide and amino acid substitutions, additions, or deletions of GLP-1 or GLP-2 molecules. Also encompassed within the term “variant(s)” are chemically modified natural and synthetic GLP-1 or GLP-2 molecules.

As used herein, the term “analog” or “analogs” as used herein refers to a polypeptide that possesses a similar or identical function to a GLP polypeptide or a fragment of a GLP polypeptide, but does not necessarily comprise a similar or identical amino acid sequence of a GLP polypeptide or a fragment of a GLP polypeptide, or possess a similar or identical structure of a GLP polypeptide or a fragment of a GLP polypeptide. A polypeptide that has a similar amino acid sequence refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a GLP polypeptide or a fragment of a GLP polypeptide described herein; (b) a polypeptide encoded by a nucleotide sequence that hybridizes under stringent conditions to a nucleotide sequence encoding a GLP polypeptide or a fragment of a GLP polypeptide described herein of at least 10 amino acid residues, at least 15 amino acid residues, at least 20 amino acid residues, at least 25 amino acid residues, or at least 30 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a GLP polypeptide or a fragment of a GLP polypeptide described herein. A polypeptide with similar structure to a GLP polypeptide or a fragment of a GLP polypeptide described herein refers to a polypeptide that has a similar secondary, tertiary or quaternary structure of a GLP polypeptide or a fragment of a GLP polypeptide described herein. The structure of a polypeptide can determined using methods known to those skilled in the art, including but not limited to, X-ray crystallography, nuclear magnetic resonance, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389 3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (e.g., http://www.ncbi.nlm.nib.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “fragment” or “fragments” as used herein refers to a peptide or polypeptide having an amino acid sequence of at least 10 contiguous amino acid residues, at least 15 contiguous amino acid residues, at least 20 contiguous amino acid residues, at least 25 contiguous amino acid residues, or at least 30 contiguous amino acid residues of the amino acid sequence of a GLP polypeptide.

As used herein, the phrase “GLP activator” or “GLP activators” refers to any molecule or compound that increases the activity of GLP in a patient. The invention encompasses, e.g., GLP agonists, GLP receptor agonists, agonist of the GLP signal transduction cascade, compounds that stimulate the synthesis or expression of endogenous GLP, compounds that stimulate release of endogenous GLP, and compounds that inhibit inhibitors of GLP activity (i.e., an inhibitor of a GLP antagonist).

As used herein, the term “patient” is an animal, such as, but not limited to, a cow, monkey, horse, sheep, pig, chicken, turkey, quail, cat, dog, mouse, rat, rabbit, and guinea pig, and is more preferably a mammal, and most preferably a human.

As used herein, the phrase “therapy” or “therapeutic agent” refers to any molecule, compound, or treatment that assists in the treatment of a disease, especially a bone-related disorder and a nutrition-related disorder. As such, therapy includes, but is not limited to, radiation therapy, chemotherapy, dietary therapy, physical therapy, and psychological therapy.

The term “administration” of the pharmaceutically active compounds and the pharmaceutical compositions defined herein includes systemic use, as by inhalation. In other embodiments, the administration may include injection (especially parenterally), intravenous infusion, suppositories and oral administration thereof, as well as topical application of the compounds and compositions.

In one embodiment, administration is by intranasal administration wherein the active ingredient is substantially absorbed through the nasal mucosa into systemic circulation. In another embodiment, administration is by intranasal administration wherein the active ingredient is substantially absorbed through the nasal mucosa into systemic circulation wherein substantially absorbed through the nasal mucosa into systemic circulation is wherein greater than 50% of the active ingredient is delivered through the nasal mucosa. In another embodiment, substantially absorbed through the nasal mucosa into systemic circulation is greater than 75% of the active ingredient is delivered through the nasal mucosa. In another embodiment, substantially absorbed through the nasal mucosa into systemic circulation is greater than 85% of the active ingredient is delivered through the nasal mucosa. In another embodiment, substantially absorbed through the nasal mucosa into systemic circulation is greater than 90% of the active ingredient is delivered through the nasal mucosa. In another embodiment, substantially absorbed through the nasal mucosa into systemic circulation is greater than 95% of the active ingredient is delivered through the nasal mucosa.

“Ameliorate” or “amelioration” means a lessening of the detrimental effect or severity of the disease in the subject receiving therapy, the severity of the response being determined by means that are well known in the art.

By “compatible” herein is meant that the components of the compositions which comprise the present invention are capable of being commingled without interacting in a manner which would substantially decrease the efficacy of the pharmaceutically active compound under ordinary use conditions.

The terms “effective amount” or “pharmaceutically effective amount” refer to a nontoxic but sufficient amount of the agent to provide the desired biological result. That result can be reduction and/or alleviation of the signs, symptoms, or causes of a disease, such as neural diseases and malignant hyperthermia, or any other desired alteration of a biological system. Such amounts are described below. An appropriate “effective” amount in any individual case may be determined by one of ordinary skill in the art using routine experimentation.

As used herein, the term “excipient” means the substances used to formulate active pharmaceutical ingredients (API) into pharmaceutical formulations; in a preferred embodiment, an excipient does not lower or interfere with the primary therapeutic effect of the API. Preferably, an excipient is therapeutically inert. The term “excipient” encompasses carriers, diluents, vehicles, solubilizers, stabilizers, bulking agents, acidic or basic pH-adjusting agents and binders. Excipients can also be those substances present in a pharmaceutical formulation as an indirect or unintended result of the manufacturing process. Preferably, excipients are approved for or considered to be safe for human and animal administration, i.e., GRAS substances (generally regarded as safe). GRAS substances are listed by the Food and Drug administration in the Code of Federal Regulations (CFR) at 21 CFR 182 and 21 CFR 184, incorporated herein by reference.

The term “formulation” can include the addition of pharmaceutically acceptable excipients, diluents, or carriers and pH adjusting agents.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

As used herein, a “pharmaceutically acceptable carrier” is a material that is nontoxic and generally inert and does not affect the functionality of the active ingredients adversely. Examples of pharmaceutically acceptable carriers are well known and they are sometimes referred to as dilutents, vehicles or excipients. The carriers may be organic or inorganic in nature. Examples of pharmaceutically acceptable carriers that may be present in the present lyophilized formulations may be gelatin, lactose, starch, cocoa butter, dextrose, sucrose, sorbitol, mannitol, gum acacia, alginates, cellulose, talc, magnesium stearate, polyoxyethylene sorbitan monolaurate, polyvinylpyro-lidone (PVP) and other commonly used pharmaceutical carriers. In one embodiment, the pharmaceutical carrier comprises mannitol. In addition, the formulation may contain minor amounts of pH adjusting agents such as sodium hydroxide (NaOH) additives such as flavoring agents, coloring agents, thickening or gelling agents, emulsifiers, wetting agents, buffers, stabilizers, and preservatives such as antioxidants.

By “physiological pH” or a “pH in the physiological range” is meant a pH in the range of approximately 7.2 to 8.0 inclusive, more typically in the range of approximately 7.2 to 7.6 inclusive.

The term “pharmaceutical composition” as used herein shall mean a composition that is made under conditions such that it is suitable for administration to humans, e.g., it is made under GMP conditions and contains pharmaceutically acceptable excipients, e.g., without limitation, stabilizers, pH adjusting agents such as NaOH, bulking agents, buffers, carriers, diluents, vehicles, solubilizers, and binders. As used herein pharmaceutical composition includes but is not limited to a pre-lyophilization solution or dispersion as well as a liquid form ready for injection or infusion after reconstitution of a lyophilized preparation.

A “pharmaceutical dosage form” as used herein means the pharmaceutical compositions disclosed herein being in a container and in an amount suitable for reconstitution and administration of one or more doses, typically about 1-2, 1-3, 1-4, 1-5, 1-6, 1-10, or about 1-20 doses.

As used herein, the term “subject” encompasses mammals and non-mammals. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or sex.

As used herein, the terms “treating” or “treatment” of a disease include preventing the disease, i.e. preventing clinical symptoms of the disease in a subject that may be exposed to, or predisposed to, the disease, but does not yet experience or display symptoms of the disease; inhibiting the disease, i.e., arresting the development of the disease or its clinical symptoms, or relieving the disease, i.e., causing regression of the disease or its clinical symptoms.

The term “treatment of alcoholism” comprises the amelioration, reduction or cessation of the desire for and habit of consuming alcoholic drinks, the treatment of alcohol dependence and the treatment of abstinence syndrome.

The term “heavy drinking” means consumption of alcohol that result in rapid intoxication, or a rapid increase in alcohol concentration in the blood to levels that would normally result in intoxication.

The term “excessive drinking” means consumption of alcohol for an extended period of time that results in intoxication that lasts longer than twice the time it would normally take the animal to reduce the amount of alcohol in the blood to below intoxicating levels.

As used herein, the phrase “isolated polypeptide or peptide” refers to a polypeptide or peptide that is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein, peptide, or fragment thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest. In preferred embodiments, purified or isolated preparations will lack any contaminating proteins from the same animal from which the protein is normally produced, as can be accomplished by recombinant expression of, for example, a human protein in a non-human cell.

As used herein, the phrase “isolated nucleic acid molecule” refers to a nucleic acid molecule which is separated from other nucleic acid molecules which are present in the natural source of the nucleic acid molecule. Preferably, an isolated nucleic acid molecule is free of sequences (preferably protein encoding sequences) which naturally flank the nucleic acid (i.e., sequences located at the 5′ and 3′ ends of the nucleic acid) in the genomic DNA of the organism from which the nucleic acid is derived. In other embodiments, the isolated nucleic acid is free of intron sequences. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an isolated nucleic acid molecule, such as a cDNA molecule, can be substantially free of other cellular material, or culture medium when produced by recombinant techniques, or substantially free of chemical precursors or other chemicals when chemically synthesized. In one embodiment, the nucleic acid molecules of the invention comprise a contiguous open reading frame encoding a polypeptide of the invention.

As used herein, the phrase “hybridizes under stringent conditions” is intended to describe conditions for hybridization and washing under which nucleotide sequences at least 60% (65%, 70%, 75%, 80%, or preferably 85% or more) identical to each other typically remain hybridized to each other. Such stringent conditions are known to those skilled in the art and can be found in Current Protocols in Molecular Biology, John Wiley & Sons, N.Y. (1989), 6.3.1 6.3.6, which describes aqueous and non-aqueous methods, either of which can be used. Another preferred, non-limiting example of stringent hybridization conditions are hybridization in 6× sodium chloride/sodium citrate (SSC) at about 45° C., followed by one or more washes in 2.0×SSC at 50° C. (low stringency) or 0.2×SSC, 0.1% SDS at 50-65° C. (high stringency). Another preferred example of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions are hybridization in 6× sodium chloride/sodium citrate (SSC) at about 45° C., followed by one or more washes in 0.2×SSC, 0.1% SDS at 60° C. Preferably, stringent hybridization conditions are hybridization in 6× sodium chloride/sodium citrate (SSC) at about 45° C., followed by one or more washes in 0.2×SSC, 0.1% SDS at 65° C. Particularly preferred stringency conditions (and the conditions that should be used if the practitioner is uncertain about what conditions should be applied to determine if a molecule is within a hybridization limitation of the invention) are 0.5M Sodium Phosphate, 7% SDS at 65° C., followed by one or more washes at 0.2×SSC, 1% SDS at 65° C. In one embodiment, an isolated nucleic acid molecule of the invention that hybridizes under stringent conditions to the sequence of the GLP nucleic acid, or a complement thereof, corresponds to a naturally-occurring nucleic acid molecule. As used herein, a “naturally occurring” nucleic acid molecule refers to an RNA or DNA molecule having a nucleotide sequence that occurs in nature (e.g. encoding a natural protein).

The GLP molecules and GLP activators disclosed herein are useful for treating or preventing addictive and compulsive diseases and disorders, particular alcohol-related diseases and disorders, disclosed herein.

The GLP activators of the present invention is effective against various alcohol and drug dependency diseases against which a GLP activator preparation is effective.

In accordance with the invention, the present compositions and methods can be used to intercede upstream or downstream in the signal transduction cascade involved in GLP action to treat various alcohol and drug dependency diseases. In one embodiment, the synthesis or release of endogenous GLP can be stimulated. In another embodiment, the endogenous synthesis or release of another molecule active in the cascade downstream from GLP, (e.g., a molecule produced in response to GLP binding to a receptor), can be stimulated.

Accordingly, the methods and compositions of the invention are useful for preventing, treating, diagnosing, or monitoring the progression various alcohol and drug dependency diseases disclosed herein.

The GLP molecules can be used in the present methods and compositions for treating or preventing various alcohol and drug dependency diseases.

In one embodiment, the GLP molecule is a GLP nucleic acid encoding a GLP polypeptide, peptide, or fragment thereof. The GLP nucleic acid is, for example, a full-length cDNA, cDNA corresponding to a protein coding region, RNA, mRNA, oligonucleotide, consensus sequence, motif, restriction fragment, antisense molecule, ribozyme, or a molecule encoding a protein domain.

In another embodiment, the GLP molecule is a GLP polypeptide or peptide, or fragment thereof. The GLP polypeptide or peptide is, for example, a full-length protein, receptor binding domain, catalytic domain, signal sequence, or protein motif.

Moreover, any GLP molecule that contains additional nucleic acid or amino acid residues, or has nucleic acids or amino acids deleted from it can be used in the present methods and compositions of the invention. Additionally, GLP molecules of the invention may contain substituted nucleic acids or amino acids. In one embodiment, the GLP variant has enhanced activity compared to native human GLP-1. For example, such GLP variants can exhibit enhanced serum stability, enhanced receptor binding, or enhanced signal transducing activity. Amino acid modifications, substitutions, additions, or truncations that render a GLP peptide resistant to oxidation or degradation are contemplated by the present invention. In a preferred embodiment, the GLP variants are derived from human or rat GLP sequences.

Molecules contemplated as GLP peptides, in accordance with the present invention are known in the art (See, e.g., U.S. Pat. No. 5,990,077; International Patent Application Nos. WO 00/34331 and WO 00/34332). For example, International Patent Application Nos. WO 00/34331 and WO 00/34332 disclose analogues of GLP-1 such as (Aib^{8,35})hGLP-1(7-36)NH₂(SEQ ID NO: 14) (see U.S. Pat. No. 6,903,186), and (Aib^{8,13}-Ala³⁵)hGLP-1(7-36)NH₂ (SEQ ID NO: 15). And U.S. Pat. No. 5,990,077, discloses forms of GLP-1 and the pharmaceutically acceptable acid salts thereof, that conform to the general formula: R1-[Y]m-His-Ala-Asp-Gly-Ser-Phe-Ser-Asp-Glu-Met-Asn-Thr-aa1-Leu-Ala-aa2-L-eu-Ala-aa3-Arg-Asp-Phe-Ile-Asn-Trp-Leu-aa4-aa5-Thr-Lys-Ile-Thr-Asp-[X]-n-R2

wherein aa refers to an amino acid residue that is synthetic or genetically encoded, and;
aa1 is a neutral/polar/large/nonaromatic residue such as Ile or Val;
aa2 is a neutral/polar residue such as Asn or Ser;
aa3 is a neutral residue such as Ala or Thr;
aa4 is a neutral/polar/large/nonaromatic residue such as Ile or Leu;
aa5 is a neutral or basic residue such as Gln or His;

### X is Arg, Lys, Arg-Lys or Lys-Lys;

### Y is Arg or Arg-Arg;

m is 0 or 1;
n is 0 or 1;
R1 is H or an N-terminal blocking group; and
R2 is OH or a C-terminal blocking group (SEQ ID NO: 16). (see U.S. Pat. No. 5,990,077)

The designation “NH₂” in hGLP-1(7-36)NH₂ indicates that the C-terminus of the peptide is amidated, hGLP-1 (7-36) means that the C-terminus is the free acid. Aib means α-aminoisobutyric acid.

GLP-1

The term “GLP-1”, or glucagon-like peptide, includes GLP-1 mimetics and its biologically active analogues as used in the context of the present invention, and can be comprised of glucagon-like peptides and related peptides and analogs of glucagon-like peptide-1 that bind to a glucagon-like peptide-1 (GLP-1) receptor protein such as the GLP-1 (7-36) amide receptor protein and has a corresponding biologically effect on insulin secretion as GLP-1 (7-36) amide, which is a native, biologically active form of GLP-1. GLP-1 can be comprised of GLP-1 (glucagon-like peptide-1) receptor agonists and analogs such as Byetta (exenatide), Victoza (liraglutide), CJC-1131(a GLP-1-albumin drug affinity complex; DAC), ZP10 (an exendin-4 derivative; AVE-0010), BIM51077 (a human GLP-1 derivative; Taspoglutide), LY315902 (a DPP-IV-resistant GLP-1 analogue), LY307161 SR (a sustained release formulation of a GLP-1 analog), LY2199265 (an Fc immunoglobulin fusion protein), LY2428757 (a pegylated GLP-1 molecule) and NN9535 (a human GLP-1R agonist). In one embodiment, the GLP-1 is a receptor agonist selected from the group consisting of exenatide, liraglutide, taspoglutide, albiglutide, and lixisenatide. In another embodiment, the GLP-1 is a DPP-4 inhibitors selected from the group consisting of sitagliptin, vildagliptin, saxagliptin, linagliptin, dutogliptin, gemigliptin, alogliptin, and berberine.

See Goke, B. and Byrne, M, Diabetic Medicine. 1996, 13:854-860. The GLP-1 receptors are cell-surface proteins found, for example, on insulin-producing pancreatic β-cells. Glucagon-like peptides and analogues will include species having insulinotropic activity and that are agonists of, i.e. activate, the GLP-1 receptor molecule and its second messenger activity on, inter alia, insulin producing β-cells. Agonists of glucagon-like peptide that exhibit activity through this receptor have been described: EP 0708179A2; Hjorth, S. A. et al., J. Biol. Chem. 269 (48):30121-30124 (1994); Siegel, E. G. et al. Amer. Diabetes Assoc. 57th Scientific Sessions, Boston (1997); Hareter, A. et al. Amer. Diabetes Assoc. 57th Scientific Sessions, Boston (1997); Adelhorst, K. et al. J. Biol. Chem. 269(9):6275-6278 (1994); Deacon C. F. et al. 16th International Diabetes Federation Congress Abstracts, Diabetologia Supplement (1997); Irwin, D. M. et al., Proc. Natl. Acad. Sci. USA. 94:7915-7920 (1997); Mosjov, S. Int. J. Peptide Protein Res. 40:333-343 (1992). Glucagon-like molecules include polynucleotides that express agonists of GLP-1, i.e. activators of the GLP-1 receptor molecule and its secondary messenger activity found on, inter alia, insulin-producing β-cells. GLP-1 mimetics that also are agonists of β-cells include, for example, chemical compounds specifically designed to active the GLP-1 receptor. Recent publications disclose Black Widow GLP-1 and Ser² GLP-1, see G. G. Holz, J. F. Hakner/Comparative Biochemistry and Physiology, Part B 121 (1998)177-184 and Ritzel, et al., A Synthetic glucagon-like peptide-1 analog with improved plasma stability, J. Endocrinol 1998 October; 159(1): 93-102. Glucagon-like peptide-1 antagonists are also known, for example see e.g. Watanabe, Y. et al., J. Endocrinol. 140(1):45-52 (1994), and include exendin (9-39) amine, an exendin analog, which is a potent antagonist of GLP-1 receptors (see, e.g. WO97/46584). Other compounds include the GLP-1 receptor agonists described in published application WO/2006/121860.

Further embodiments include chemically synthesized glucagon-like polypeptides as well as any polypeptides or fragments thereof which are substantially homologous. “Substantially homologous,” which can refer both to nucleic acid and amino acid sequences, means that a particular subject sequence, for example, a mutant sequence, varies from a reference sequence by one or more substitutions, deletions, or additions, the net effect of which does not result in an adverse functional dissimilarity between reference and subject sequences. For purposes of the present invention, sequences having greater than 50 percent homology, and preferably greater than 90 percent homology, equivalent biological activity in enhancing 1′-cell responses to plasma glucose levels, and equivalent expression characteristics are considered substantially homologous. For purposes of determining homology, truncation of the mature sequence should be disregarded. Sequences having lesser degrees of homology, comparable bioactivity, and equivalent expression characteristics are considered equivalents.

Mammalian GLP peptides and glucagon are encoded by the same gene. In the ileum the phenotype is processed into two major classes of GLP peptide hormones, namely GLP-1 and GLP-2. There are four GLP-1 related peptides known which are processed from the phenotypic peptides. GLP-1 (1-37) has the sequence His Asp Glu Phe Glu Arg His Ala Glu Gly Thr Phe Thr Ser Asp Val Ser Ser Tyr Leu Glu Gly Gln Ala Ala Lys Glu Phe Ile Ala Trp Leu Val Lys Gly Arg Gly (SEQ ID NO:1). GLP-1 (1-37) is amidated by post-translational processing to yield GLP-1 (1-36) NH₂ which has the sequence His Asp Glu Phe Glu Arg His Ala Glu Gly Thr Phe Thr Ser Asp Val Ser Ser Tyr Leu Glu Gly Gln Ala Ala Lys Glu Phe Ile Ala Trp Leu Val Lys Gly Arg (NH₂) (SEQ ID NO:2); or is enzymatically processed to yield GLP-1 (7-37) which has the sequence His Ala Glu Gly Thr Phe Thr Ser Asp Val Ser Ser Tyr Leu Glu Gly Gln Ala Ala Lys Glu Phe Ile Ala Trp Leu Val Lys Gly Arg Gly (SEQ ID NO:3). GLP-1 (7-37) can also be amidated to yield GLP-1 (7-36) amide which is the natural form of the GLP-1 molecule, and which has the sequence His Ala Glu Gly Thr Phe Thr Ser Asp Val Ser Ser Tyr Leu Glu Gly Gln Ala Ala Lys Glu Phe Ile Ala Trp Leu Val Lys Gly Arg (NH₂) (SEQ ID NO:4) and in the natural form of the GLP-1 molecule.

Intestinal L cells secrete GLP-1 (7-37) (SEQ ID NO:3) and GLP-1(7-36)NH₂ (SEQ ID NO:4) in a ratio of 1 to 5, respectively. These truncated forms of GLP-1 have short half-lives in situ, i.e., less than 10 minutes, and are inactivated by an aminodipeptidase IV to yield Glu Gly Thr Phe Thr Ser Asp Val Ser Ser Tyr Leu Glu Gly Gln Ala Ala Lys Glu Phe Ile Ala Trp Leu Val Lys Gly Arg Gly (SEQ ID NO:5) and Glu Gly Thr Phe Thr Ser Asp Val Ser Ser Tyr Leu Glu Gly Gln Ala Ala Lys Glu Phe Ile Ala Trp Leu Val Lys Gly Arg (NH₂) (SEQ ID NO:6), respectively. The peptides Glu Gly Thr Phe Thr Ser Asp Val Ser Ser Tyr Leu Glu Gly Gln Ala Ala Lys Glu Phe Ile Ala Tip Trp Leu Val Lys Gly Arg Gly (SEQ ID NO:5) and Glu Gly Thr Phe Thr Ser Asp Val Ser Ser Tyr Leu Glu Gly Gln Ala Ala Lys Glu Phe Ile Ala Trp Leu Val Lys Gly Arg (NH₂) (SEQ ID NO:6), have been speculated to affect hepatic glucose production, but do not stimulate the production or release of insulin from the pancreas.

There are six peptides in Gila monster venoms that are homologous to GLP-1. Their sequences are compared to the sequence of GLP-1 in Table 1.

TABLE 1

| | |
|---|---|
| a | H A E G T F T S D V S S Y L E G Q A A K E F I A W L V K G R (NH₂) |
| b | H S D G T F T S D L S K Q M E E E A V R L F I E W L K N G G P S S G A P P P S (NH₂) |
| c | D L S K Q M E E E A V R L F I E W L K N G G P S S G A P P P S (NH₂) |
| d | H G E G T F T S D L S K Q M E E E A V R L F I E W L K N G G P S S G A P P P S (NH₂) |
| e | H S D A T F T A E Y S K L L A K L A L Q K Y L E S I L G S S T S P R P P S S (NH₂) |
| f | H S D A T F T A E Y S K L L A K L A L Q K Y L E S I L G S S T S P R P P S (NH₂) |
| g | H S D A I F T E E Y S K L L A K L A L Q K Y L A S I L G S R T S P P P (NH₂) |
| h | H S D A I F T Q Q Y S K L L A K L A L Q K Y L A S I L G S R T S P P P (NH₂) |

a = GLP-1(7-36)amide (SEQ. ID NO: 4).
b = exendin 3 (SEQ. ID NO: 7).
c = exendin 4 (9-39(NH₂) (SEQ. ID NO: 8).
d = exendin 4 (SEQ. ID NO: 9).
e = helospectin I (SEQ. ID NO: 10).
f = helospectin II (SEQ. ID NO: 11).
g = helodermin (SEQ. ID NO: 12).
h = Q⁸, Q⁹ helodermin (SEQ. ID NO: 13).

The peptides c and h are derived from b and g, respectively. All 6 naturally occurring peptides (a, b, d, e, f and g) are homologous in positions 1, 7, 11 and 18. GLP-1 and exendins 3 and 4 (a, b and d) are further homologous in positions 4, 5, 6, 8, 9, 15, 22, 23, 25, 26 and 29. In position 2, A, S and G are structurally similar. In position 3, residues D and E (Asp and Glu) are structurally similar. In positions 22 and 23 F (Phe) and I (Ile) are structurally similar to Y (Tyr) and L (Leu.), respectively. Likewise, in position 26 L and I are structurally equivalent.

Thus, of the 30 residues of GLP-1, exendins 3 and 4 are identical in 15 positions and equivalent in 5 additional positions. The only positions where radical structural changes are evident are at residues 16, 17, 19, 21, 24, 27, 28 and 30. Exendins also have 9 extra residues at the carboxyl terminus.

“GLP-1 molecule” further denotes biologically active variants, analogs, and derivatives of GLP-1 peptides. “Biologically active,” in this context, means having GLP-1(7-36) biological activity, but it is understood that the variant, analog, or derivative can be either less or more potent than native GLP-1(7-36)amide, a native, biologically active form of GLP-1. See Goke & Byrne, Diabetic Medicine. 13; 854 (1996). GLP-1 molecules of the present invention include polynucleotides that express agonists of GLP-1 (i.e., activators of the GLP-1 receptor molecule and its secondary messenger activity found on, inter alia, insulin-producing β-cells). GLP-1 mimetics that also are agonists of β-cells include, for example, chemical compounds specifically designed to activate the GLP-1 receptor. Included as GLP-1 molecules are any molecules, whether they be peptides, peptide mimetics, or other molecules that bird to or activate a GLP-1 receptor, such as the GLP-1(7-36)amide receptor, and its second messenger cascade. GLP-1 molecules include species having insulinotropic activity and that are agonists of (i.e., activate), the GLP-1 receptor molecule and its second messenger activity on, inter alia, insulin producing β-cells.

“GLP-1 molecules” also include peptides that are encoded by polynucleotides that express biologically active GLP-1 variants, as defined herein. Also included in the present invention are GLP-1 molecules that are peptides containing one or more amino acid substitutions, additions or deletions, compared with GLP-1(7-36)amide. In one embodiment, the number of substitutions, deletions, or additions is 30 amino acids or less, 25 amino acids or less, 20 amino acids or less, 15 amino acids or less, 10 amino acids or less, 5 amino acids or less or any integer in between these amounts. In one aspect of the invention, the substitutions include one or more conservative substitutions. A “conservative” substitution denotes the replacement of an amino acid residue by another, biologically active similar residue. Examples of conservative substitution include the substitution of one hydrophobic residue, such as isoleucine, valine, leucine, or methionine for another, or the substitution of one polar residue for another, such as the substitution of arginine for lysine, glutamic for aspartic acids, or glutamine for asparagine, and the like. The following table lists illustrative, but non-limiting, conservative amino acid substitutions.

TABLE

| Original Residue | Exemplary Substitutions |
|---|---|
| ALA | SER, THR |
| ARG | LYS |
| ASN | HIS, SER |
| ASP | GLU, ASN |
| CYS | SER |
| GLN | ASN, HIS |
| GLU | ASP, GLU |
| GLY | ALA, SER |
| HIS | ASN, GLN |
| ILE | LEU, VAL, THR |
| LEU | ILE, VAL |
| LYS | ARG, GLN, GLU, THR |
| MET | LEU, ILE, VAL |
| PHE | LEU, TYR |
| SER | THR, ALA, ASN |
| THR | SER, ALA |
| TRP | ARG, SER |
| TYR | PHE |
| VAL | ILE, LEU, ALA |
| PRO | ALA |

It is further understood that GLP-1 peptide variants include the above described peptides which have been chemically derivatized or altered, for example, peptides with non-natural amino acid residues (e.g., taurine residue, β- and γ-amino acid residues and D-amino acid residues), C-terminal functional group modifications such as amides, esters, and C-terminal ketone modifications and N-terminal functional group modifications such as acylated amines, Schiff bases, or cyclization, such as found, for example, in the amino acid pyroglutamic acid.

In preferred embodiments, the present invention also encompasses a polynucleotide that comprises a polypeptide that encodes at least about 29 contiguous amino acids of SEQ ID NO:2. Preferably, the polypeptides and/or polypeptides encoded by said polynucleotides retain GLP-1 activity. In this context, the term “about” may be construed to mean 1, 2, 3, 4, 5, 6, 7, 8, 9, 10, 11, 12, 13, 14, 15, 16, 17, 18, 19, or 20 amino acids beyond the N-Terminus and/or C-terminus of the above referenced amino acid locations.

Many variants of GLP-1 are known in the art such as, for example, Gln⁹-GLP-1 (SEQ ID NO: 17), D-Gln⁹-GLP-1 (SEQ ID NO: 18), acetyl-Lys⁹-GLP-1 (SEQ ID NO: 19), Thr¹⁶-Lys¹⁸-GLP-1 (SEQ ID NO: 20), and Lys¹⁸-GLP-1 (SEQ ID NO: 21) as listed in WO 91/11457. Acid addition salts, carboxylate salts, lower alkyl esters, and amides of GLP-1 variants, many of which are disclosed in the art, are also contemplated by the invention.

Variants of GLP-1 can be obtained by fragmenting a naturally occurring sequence, or can be synthesized based upon knowledge of the DNA, RNA, or amino acid sequence of a native GLP-1. Processes for preparing these variants are known to those of ordinary skill in the art (See, e.g., WO 91/11457; U.S. Pat. Nos. 5,118,666, 5,120,712, and 5,512,549). For example, variants can be prepared using standard solid-phase techniques for the synthesis of peptides. As is generally known, peptides of the requisite length can be prepared using commercially available equipment and reagents following the manufacturers' instructions for blocking interfering groups, protecting the amino acid to be reacted, coupling, deprotection, and capping of unreacted residues. Suitable equipment can be obtained, for example, from Applied BioSystems in Foster City, Calif., or Biosearch Corporation in San Raphael, Calif. It is also possible to obtain fragments of GLP-1, by fragmenting the naturally occurring amino acid sequence, using, for example, a proteolytic enzyme. Further, it is possible to obtain the desired fragments of the GLP-1 through the use of recombinant DNA technology. The basic steps in recombinant production are: a) isolating a natural DNA sequence encoding GLP-1 or constructing a synthetic or semi-synthetic DNA coding sequence for GLP-1, b) placing the coding sequence into an expression vector in a manner suitable for expressing proteins either alone or as a fusion proteins, c) transforming an appropriate eukaryotic or prokaryotic host cell with the expression vector, d) culturing the transformed host cell under conditions that will permit expression of a GLP-1 intermediate, and e) recovering and purifying the recombinantly produced protein.

In one embodiment, the GLP molecule is a GLP-1 variant having enhanced insulin-stimulating properties as disclosed in U.S. Pat. No. 5,545,618. The variants can be GLP-1(7-34) (SEQ ID NO: 22); (7-35) (SEQ ID NO: 23) (7-36) (SEQ ID NO: 24) or (7-37) (SEQ ID NO: 25) human peptide or the C-terminal amidated forms thereof. The native peptides have the amino acid sequence (wherein the first amino acid below (i.e., His) is at position 7): His-Ala-Gln-Gly-Thr-Phe-Thr-Ser-Asp-Val-Ser-Ser-Tyr-Leu-Gln-Gly-Glu-Ala-Ala-Lys-Gln-Phe-Ile-Ala-Trp-Leu-Val-Lys-(Gly)-(Arg)-(Gly) (SEQ ID NO: 26) wherein (Gly), (Arg), and (Gly) are present or absent depending on indicated chain length.

The variants have the foregoing sequence, or the C-terminal amide thereof, with at least one modification selected from the group consisting of:

(a) (SEQ ID NO: 27) substitution of a neutral amino acid, arginine, or a D form of lysine for lysine at position 26 and/or 34 and/or a neutral amino acid, lysine, or a D form of arginine for arginine at position 36;

(b) (SEQ ID NO: 28) substitution of an oxidation-resistant amino acid for tryptophan at position 31;

(c) (SEQ ID NO: 29) substitution according to at least one of: Tyr for Val at position 16; Lys for Ser at position 18; Asp for Gln at position 21; Ser for Gly at position 22; Arg for Glu at position 23; Arg for Ala at position 24; and Glu for Lys at position 26;

(d) (SEQ ID NO: 30) a substitution comprising at least one of: an alternative small neutral amino acid for A. at position 8; an alternative acidic amino acid or neutral amino acid for Glu at position 9; an alternative neutral amino acid for Gly at position 10; and an alternative acidic amino acid for Asp at position 15; and

(e) (SEQ ID NO: 31) substitution of an alternative neutral amino acid or the D or N-acylated or alkylated form of histidine for histidine at position 7.

In another embodiment, the GLP molecule is a GLP-1 variant having enhanced resistance to degradation as compared to native GLP-1. Enhanced resistance to degradation can result in longer bioavailability. In a specific embodiment, the GLP-1 variant demonstrates both enhanced activity and enhanced stability.

The particular form of GLP-1 selected for inhibiting alcohol and drug dependency can be prepared by a variety of techniques well known for generating peptide products. As described by Buhl et al, supra, porcine GLP-1 isolation and purification is achieved from acid-ethanol extracts of ileal mucosa by a combination of size selection and HPLC-based fractionation, with the aid of antibody raised against synthetic proglucagon 126 159, to monitor work-up. As an alternative to GLP-1 extraction, those forms of GLP-1 that incorporate only L-amino acids can be produced reproducibly and in commercial quantities by application of recombinant DNA technology. For this purpose, DNA coding for the desired form of GLP-1 is incorporated expressibly in a microbial e.g. yeast, or other cellular host, which is then cultured under conditions appropriate for GLP-1 expression. A variety of gene expression systems have been adapted for this purpose, and typically drive expression of the desired gene from expression controls used naturally by the chosen host. Because GLP-1 does not require post translational glycosylation for its activity, its production may most conveniently be achieved in bacterial hosts such as E. coli. For such production, DNA coding for the selected GLP-1 may usefully be placed under expression controls of the lac, trp or PL genes of E. coli. As an alternative to expression of DNA coding for the GLP-1 per se, the host can be adapted to express GLP-1 as a fusion protein it which the GLP-1 is linked releasably to a carrier protein that facilitates isolation and stability of the expression product.

In an approach universally applicable to the production of a selected GLP-1, and one used necessarily to produce GLP-1 forms that incorporate non-genetically encoded amino acids and N- and C-terminally derivatized forms, the-well established techniques of automated peptide synthesis are employed, general descriptions of which appear, for example, in J. M. Stewart and J. D. Young, Solid Phase Peptide Synthesis, 2nd Edition, 1984, Pierce Chemical Company, Rockford, Ill.; and in M. Bodanszky and A. Bodanszky, The Practice of Peptide Synthesis, 1984, Springer-Verlag, N.Y.; Applied Biosystems 430A Users Manual, 1987, ABI Inc., Foster City, Calif. In these techniques, the GLP-1 is grown from its C-termitial, resin-conjugated residue by the sequential addition of appropriately protected amino acids, using either the Fmoc or tBoc protocols, as described for instance by Orskov et al, 1989, supra.

For the incorporation of N- and/or C-protecting groups protocols is conventional to solid phase peptide synthesis methods can also be applied. For incorporation of C-terminal protecting groups, for example, synthesis of the desired peptide is typically performed using, as solid phase, a supporting resin that has been chemically modified so that cleavage from the resin results in a peptide having the desired C-terminal protecting group. To provide peptides in which the C-terminus bears a primary amino protecting group, for instance, synthesis is performed using a p-methylbenzhydrylamine, (MBHA) resin so that, when peptide synthesis is completed, treatment with hydrofluoric acid releases the desired C-terminally amidated peptide. Similarly, incorporation of an N-methylamine protecting group at the C-terminus is achieved using N methylaminoethyl-derivatized DVB resin, which upon HF treatment releases peptide baring an N-methylamidated C-terminus. Protection of the C-terminus by esterification can also be achieved using conventional procedures. This entails use of resin/blocking group combination that permits release of side-chain protected peptide from the resin, to allow for subsequent reaction with the desired alcohol, to form the ester function. FMOC protecting groups, in combination with DVB resin derivatized with methoxyalkoxybenzyl alcohol or equivalent linker, can be used for this purpose, with cleavage from the support being effected by TFA in dichloromethane. Esterification of the suitably activated carboxyl function e.g. with DCC, can then proceed by addition of the desired alcohol, followed by deprotection and isolation of the esterified peptide product.

Incorporation of N-terminal protecting groups can be achieved while the synthesized peptide is still attached to the resin, for instance by treatment with suitable anhydride and nitrile. To incorporate an acetyl protecting group at the N-terminus, for instance, the resin-coupled peptide can be treated with 20% acetic anhydride in acetonitrile. The N-protected peptide product can then be cleaved from the resin, deprotected and subsequently isolated.

Once the desired peptide sequence has been synthesized, cleaved from the resin and fully deprotected, the peptide is then purified to ensure the recovery of a single oligopeptide having the selected amino acid sequence, Purification can be achieved using any of the standard approaches, which include reversed-phase high-pressure liquid chromatography (RP-HPLC) on alkylated silica columns, e.g. C4-, C8-, or C18 .about.silica. Such column fractionation is generally accomplished by running linear gradients, e.g. 10 90%, of increasing % organic solvent, e.g. acetonitrile, in aqueous buffer, usually containing a small amount (e.g. 0.1%) of pairing agent such as TFA or TEA. Alternatively, ion-exchange HPLC can be employed to separate peptide species on the basis of their charge characteristics. Column fractions are collected, and those containing peptide of the desired/required purity are optionally pooled. In one embodiment of the invention, the peptide is then treated in the established manner to exchange the cleavage acid (e.g. TFA) with a pharmaceutically acceptable acid, such as acetic, hydrochloric, phosphoric, maleic, tartaric, succinic and the likes to provide a water soluble salt of the peptide.

For administration to patients, the GLP-1 is provided, in one aspect of the invention, in pharmaceutically acceptable form, e.g., as a preparation that is sterile-filtered e.g. through a 0.22μ filter, and substantially pyrogen-free. Desirably, the GLP-1 to be formulated migrates as a single or individualized peak on HPLC, exhibits uniform and authentic amino acid composition and sequence upon analysis thereof, and otherwise meets standards set by the various national bodies which regulate quality of pharmaceutical products.

For therapeutic use, the chosen GLP-1 is formulated with a carrier that is pharmaceutically acceptable and is appropriate for delivering the peptide by the chosen route of administration. Suitable pharmaceutically acceptable carriers are those used conventionally with peptide-based drugs, such as diluents, excipients and the like. Reference may be made to “Remington s Pharmaceutical Sciences”, 17th Ed., Mack Publishing Company, Easton, Pa., 1995, for guidance on drug formulations generally. In one embodiment of the invention the compounds are formulated for administration by infusion or by injection, either sub-cutaneously or intravenously, and are accordingly utilized as aqueous solutions in sterile and pyrogen-free form and optionally buffered to a slightly acidic or physiological pH. Thus, the compounds may be administered in distilled water or, more desirably, in saline, buffered saline or 5% dextrose solution. Water solubility of these and other the GLP-1 may be enhanced, if desired, by incorporating a solubility enhancer, such as acetic acid.

For use in inhibiting the alcohol and drug dependency in a mammal including a human, the present invention provides in one of its aspects a package, in the form of a sterile-filled vial or ampoule, that contains an alcohol and drug dependency inhibiting amount of the GLP-1 analogue, in either unit dose or multi-dose amounts, wherein the package incorporates a label instructing use of its contents for the inhibition of alcohol and drug dependency. In one embodiment of the invention, the package contains the GLP-1 and the desired carrier, as an administration-ready formulation. Alternatively, and according to another embodiment of the invention, the package provides the GLP-1 in a form, such as a lyophilized form, suitable for reconstitution in a suitable carrier, such as buffered saline.

In one embodiment, the package is a sterile-filled vial or ampoule containing an injectable solution which comprises an effective amount of GLP-1 dissolved in an aqueous vehicle.

As an alternative to injectable formulations, the GLP-1 may be formulated for administration by other routes. Oral dosage forms, such as tablets, capsules and the like, can be formulated in accordance with standard pharmaceutical practice.

In one embodiment, the GLP-1 variant is resistant to cleavage by dipeptidyl peptidase-IV (DPP-IV).

In another embodiment, the GLP-1 variant has an amino acid sequence wherein an oxidatively sensitive amino acid, is replaced with an oxidatively stable amino acid residue. In another embodiment, the oxidatively sensitive amino acid is methionine (“Met”). These variants can be more stable than a native GLP-1.

In another embodiment, the GLP-1 variant has an amino acid sequence wherein an arginine is replaced with a basic amino acid (e.g., histidine or lysine).

GLP Activators

The invention also encompasses molecules that serve to increase GLP activity (GLP activators) for use in prevention and treatment of alcohol and drug dependency. For example, GLP agonists, GLP receptor agonists, agonist of the GLP signal transduction cascade, compounds that stimulate the synthesis or expression of endogenous GLP, compounds that stimulate release of endogenous GLP, and compounds that inhibit inhibitors of GLP activity (i.e., an inhibitor of a GLP antagonist) are contemplated.

In one embodiment, the GLP activator is a GLP-1 agonist as known in the art.

In a particular embodiment, GLP-1 agonists have a(n): N-terminal blocking group; and/or N-terminal extension such as Arg or Arg-Arg; and/or C-terminal blocking group; and/or C-terminal extension such as Arg or Arg-Arg.

In another embodiment, the GLP molecule useful for the invention is an inhibitor of a GLP antagonist. In a particular embodiment, the GLP antagonist is a protease. In a specific embodiment, the protease is DPP-IV.

Useful inhibitors of the GLP antagonist, DPP-W, include, but are not limited to, N-(substituted glycyl)-2-cyanopyrrolidines, N-Ala-Pro-O-(nitrobenzyl-) hydroxylamine, and .epsilon.-(4-nitro) benzoxycarbonyl-Lys-Pro. Other useful inhibitors of DPP-IV are known in the art (See, e.g., U.S. Pat. No. 5,462,928 (columns 2 4), U.S. Pat. No. 5,543,396 (column 2) and U.S. Pat. No. 6,124,305 (columns 1 2). Some examples are: X-Pro-Y-boroPro, where X and Y are chosen from any amino acid residue, and where boroPro is used to designate an α-amino boronic acid analog of proline which has the carboxyl group of proline replaced with a B(OH)₂ group; peptidyl derivatives of aromatic diesters of α-aminoalkylphosphonic acids; and N-(substituted glycyl)-2-cyanopyrrolidines.

In yet another embodiment, the inhibitor of a GLP antagonist is an antibody directed against a GLP antagonist. In a further embodiment, the inhibitor is an antibody directed against DPP-IV (See, e.g., U.S. Pat. No. 6,265,551). For example, U.S. Pat. No. 6,265,551 discloses antibodies that bind specifically to the 175 kDa form of DPPIV/CD26 but not to the 105 kDa form.

Also encompassed by the invention are nucleic acid molecules encoding GLP activators that are polypeptides. The nucleic acid is preferably found in a mammalian expression vector comprising a tumor-specific, tissue-specific, and/or inducible transcriptional regulatory sequence.

The GLP molecules or GLP activators are administered to a patient, preferably a mammal, more preferably a human, for the treatment or prevention of an alcohol and drug dependency. The GLP molecules or GLP activators of the invention can be used to treat acute or chronic forms of these conditions.

The GLP molecules and GLP activators and optionally another therapeutic agent are administered at an effective dose. The dosing and regimen most appropriate for patient treatment will vary with the disease or condition to be treated, and in accordance with the patient's weight and with other parameters.

An effective dosage and treatment protocol can be determined by conventional means, comprising the steps of starting with a low dose in laboratory animals, increasing the dosage while monitoring the effects (e.g., histology, disease activity scores), and systematically varying the dosage regimen. Several factors may be taken into consideration by a clinician when determining an optimal dosage for a given patient. Primary among these is the amount of GLP molecule normally circulating in the plasma, which, in the case of a GLP peptide, is approximately 150 pmol/ml in the resting state, and rising to approximately 225 pmol/ml after nutrient ingestion for healthy adult humans (Orskov and Holst, 1987, Scand J. Clin. Lab. Invest. 47:165). Additional factors include, but are not limited to, the size of the patient, the age of the patient, the general condition of the patient, the particular disease being treated, the severity of the disease, the presence of other drugs in the patient, and the in vivo activity of the GLP molecule.

Trial dosages would be chosen after consideration of the results of animal studies and the clinical literature. A person of ordinary skill in the art can appreciate that information such as binding constants and Ki derived from in vitro GLP binding competition assays may also be used in calculating dosages.

A typical effective human dose of a GLP molecule or GLP activator would be from about 10 μg/kg body weight/day to about 10 mg/kg/day, preferably from about 50 μg/kg/day to about 5 mg/kg/day, and most preferably about 100 μg/kg/day to 1 mg/kg/day. As analogs of the GLP molecules and GLP activators disclosed herein can be 2 to 100 times more potent than naturally occurring counterparts, a typical effective dose of such a GLP analog can be lower, for example, from about 100 ng/kg body weight/day to 1 mg/kg/day, preferably 1 μg/kg/day to 500 μg/kg/day, and even more preferably 1 μg/kg/day to 100 μg/kg/day.

In another embodiment, the effective dose of a GLP molecule or a GLP activator is less than 10 μg/kg/day. In yet another embodiment the effective dose of a GLP molecule or GLP activator is greater than 10 mg/kg/day.

The specific dosage for a particular patient, of course, has to be adjusted to the degree of response, the route of administration, the patients weight, and the patient's general condition, and is finally dependent upon the judgment of the treating physician.

Gene Therapy

Gene therapy approaches can also be used in accordance with the present invention to modulate the expression of a GLP molecule or GLP activator and accordingly, to treat or prevent an alcohol and drug dependency.

Any of the methods for gene therapy available in the art can be used in accordance with the present invention (See, e.g., Goldspiel et al., 1993, Clin. Pharm. 12:488 505; Grossman and Wilson, 1993, Curr. Opin. Genet. Devel. 3:110 114; Salmons and Gunzberg, 1993, Hum. Gene Ther. 4:129 141; Morgan and Anderson, 1993, Ann. Rev. Biochem. 62:191 217; Mulligan, 1993, Science 260:926 932; Tolstoshev, 1993, Ann. Rev. Pharmacol. Toxicol. 32:573 596; and Clowes et al., 1994, J. Clin. Invest. 93:644 651; Kiem et al., 1994, Blood 83:1467 1473, each of which is incorporated herein by reference).

Long-term effective use of a gene therapy vector to ameliorate disease in large mammals has been demonstrated. For example, administration of an AAV containing a wild-type gene to dogs suffering from Leber congenital amaurosis, a condition that results in blindness due to a mutation of a gene (RPE65) in the retinal pigment epithelium, has successfully corrected the genetic defect (Ackland et al., 2001, Nat. Genet. 28:92). Expression of the wild-type RPE65 gene was confirmed by RT PCR and restoration of function was demonstrated by electrophysiological studies of the retina, as well as by unbiased observational studies of the treated dogs. The treatment was shown to be effective for at least four months.

Intramuscular administration of an AAV encoding for factor IX to treat dogs suffering from hemophilia has also been reported (Herzog et al., 1999, Nat. Med. 5:56). Administration of AAV encoding factor IX was shown to significantly reduce clotting time in treated dogs for 17 months. Thus, such examples demonstrate that gene therapy can be used to restore lost genetic function in a large animal model using treatment methods known in the art.

Gene therapy refers to therapy performed by administering to a patient an expressed or expressible nucleic acid. Gene therapy involves introducing a gene construct to cells in tissue culture or in vivo.

The recipient's cells or heterologous cells can be engineered to express one or more of the GLP molecules and GLP activators or a combination of a GLP molecule or GLP activator and another therapeutic agent. Methods for introduction of nucleic acid sequences encoding GLP molecules or GLP activators (See, e.g., Bell et al., 1983, Nature 304: 5924) to cells in vitro include, but are not limited to, electroporation, lipofection, DEAE-Dextran transfection, calcium phosphate-mediated transfection, liposome-mediated transfer, and viral infection.

Such ex vivo treatment protocols can be used to transfer DNA into a variety of different cell types including, but not limited to, epithelial cells (U.S. Pat. No. 4,868,116; Morgan and Mulligan WO87/00201; Morgan et al., 1987, Science 237:1476 1479; Morgan and Mulligan, U.S. Pat. No. 4,980,286), endothelial cells (WO89/05345), fibroblasts (Palmer et al., 1987, Proc. Natl. Acad. Sci. 84:1055 1059; Anson et al., 1987, Mol. Biol. Med. 4:11 20; Rosenberg et al., 1988, Science 242:1575 1578; U.S. Pat. No. 4,963,489), lymphocytes (U.S. Pat. No. 5,399,346; Blaese et al., 1995, Science 270:475 480), and hematopoietic stem cells (Lim et al., 1989, Proc. Natl. Acad. Sci. 86:8892 8896; U.S. Pat. No. 5,399,346).

Accordingly, one can use gene therapy to create a cell line that produces any GLP molecule or GLP activator. Additionally, cells can be engineered to produce a GLP molecule or GLP activator alone or in combination with another agent such as, but not limited to, a peptide hormone (e.g., IGF-1, IGF-2 or growth hormone). The cells can be grown as an implant in an experimental animal or in tissue culture using techniques known in the art. Various expression vectors, including viral vectors, suitable for introduction of genetic information into human cells, can be used to incorporate the constructs encoding the GLP molecule or GLP activator and/or the other therapeutic agent. Once altered genetically, the engineered cells can then be administered to a subject using procedures known in the art.

Alternatively, one can use gene therapy to transfect the recipient's cells in vivo. Methods of administering vectors that transfect cells in vivo are known in the art. Formulations of nucleic acid for such in vivo methods can be, but are not limited to, naked DNA; nucleic acid encapsulated into liposomes or liposomes combined with viral envelope receptor proteins (Nicolau et al., 1983, Proc. Natl. Acad. Sci. 80:1068), DNA coupled to a polylysine-glycoprotein carrier complex, and nucleic acid precipitants.

Nucleic acid preparations can be introduced in vivo using any one of the techniques known in the art such as direct injection, electroporation, and particle bombardment. In addition, “gene guns” have been used for gene delivery into cells (Australian Patent No. 9068389).

Synthetic genes which result in the production of a GLP molecule or GLP activator following either in vitro or in vivo transcription and translation can be constructed using techniques well known in the art (See, e.g., Ausubel et al., 1990, Current Protocols in Molecular Biology p. 8.2.8 to 8.2.13.; Ausubel et al., 1995, Short Protocols in Molecular Biology p. 8.8 8.9, John Wiley & Sons Inc.).

A GLP antagonist can be inhibited with a GLP activator (i.e., an inhibitor of a GLP antagonist) with the use of gene therapy (e.g., antisense, ribozyme, triple helix molecules, and/or recombinant antibodies). In this embodiment, introduction of the GLP activator into a patient results in a decrease in the respective GLP-antagonist-gene expression and/or GLP antagonist protein levels. Techniques for the production and use of antisense, ribozyme, and/or triple helix molecules are well known to those of skill in the art, and in accordance with the present invention.

The present invention encompasses vectors comprising a nucleic acid encoding a polypeptide or peptide GLP molecule or GLP activator of the invention. In one embodiment, a nucleic acid encoding a GLP molecule or GLP activator to be introduced for purposes of gene therapy comprises an inducible promoter operably linked to the coding region, such that expression of the nucleic acid can be controlled using an appropriate inducer or inhibitor of transcription. In another embodiment, the vector contains a promoter, which expresses the cloned construct constitutively. In a further embodiment, the promoter can be down-regulated using a suppressor molecule. Alternatively, the vector contains a promoter, such that an inducing molecule initiates or increases expression of the cloned nucleic acid. In a preferred embodiment, the vector contains a cell-specific promoter. In another preferred embodiment, the vector contains a disease-specific promoter, such that expression is largely limited to diseased tissues or tissues surrounding diseased tissues.

Usually, the method of cellular introduction also comprises the transfer of a selectable marker to the cells, after which the cells are placed under selection to isolate the cells that have taken up and that express the transferred gene. These transfected cells can be administered to a patient.

Several methods have been developed for delivering the nucleic acid molecules to target cells or target tissues. Accordingly, the nucleic acid molecules can be delivered in vivo or ex vivo to target cells. In one embodiment, an expression construct can be delivered directly into a patient. In a particular embodiment, the nucleic acid molecules of the GLP molecule or GLP activator can be injected directly into the target tissue or cell derivation site. Alternatively, a patient's cells are first transfected with an expression construct in vitro, after which the transfected cells are administered back into the subject (i.e., ex vivo gene therapy).

In one embodiment, a vector is introduced in vivo such that it is taken up by a cell and directs the transcription of a nucleic acid of the invention. Such a vector can remain episomal or can become chromosomally integrated. Expression vectors can be plasmid, viral, or others known in the art, that can be used to replicate and/or express the cloned nucleotide sequence encoding a GLP nucleic acid in a target mammalian cell. A variety of expression vectors useful for introducing into cells the nucleic acid molecules are well known in the art (e.g., pCI, pVPack, pCMV, pSG5). Expression constructs can be introduced into target cells and/or tissues of a subject using vectors which including but not limited to, adenovirus, adeno-associated virus, retrovirus and herpes virus vectors, in addition to other particles that introduce DNA into cells, such as liposomes.

In a particular embodiment, the nucleic acid molecules can be introduced into the target tissue as an implant, for example, in a polymer formulation (See, e.g., U.S. Pat. No. 5,702,717). In another embodiment, the nucleic acid molecules can be targeted to the desired cells or tissues.

A nucleic acid sequence can be expressed using any promoter known in the art capable of expression in mammalian, preferably human cells. Such promoters can be inducible or constitutive. These promoters include, but are not limited to, the SV40 early promoter region (Bernoist and Chambon, 1981, Nature 290:304 310), the promoter contained in the 3′ long terminal repeat of Rous sarcoma virus (Yamamoto et al., 1980, Cell 22:787 797), the herpes thymidine kinase promoter (Wagner et al., 1981, Proc. Natl. Acad. Sci. 78:1441 1445), and the regulatory sequences of the metallothionein gene (Brinster et al., 1982, Nature 296:39 42). Tissue-specific promoters include the promoter region of osteocalcin.

In one embodiment, in which recombinant cells are used in gene therapy, nucleic acid sequences encoding polypeptides of the invention are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention, such as, but not limited to, hematopoietic cells, neuronal progenitor cells, hepatic progenitor cells, osteoblasts, and fetal stem cells (See, e.g., PCT Publication WO 94/08598; Stemple and Anderson, 1992, Cell 71:973 985; Pittelkow and Scott, 1986, Mayo Clinic Proc. 61:771; Rheinwald, 1980, Meth Cell Bio. 21A:229).

In other embodiments, the nucleic acid of the invention can include other appended groups such as peptides (e.g., for targeting host cell receptors in vivo), or agents facilitating transport across the cell membrane (see, e.g., Letsinger et al., 1989, Proc. Natl. Acad. Sci. 86:6553 6556; Lemaitre et al., 1987, Proc. Natl. Acad. Sci. 84:648 652; PCT Publication No. WO 88/09810) or the blood-brain barrier (See, e.g., PCT Publication No. WO 89/10134). For example, PCT Publication No. WO 88/09810 discloses nucleic acid conjugates comprising a relatively short oligonucleotide sequence, a linking group, and group which modifies the hydrophilic lipophilic balance to provide an amphiphillic product that aids in the transport of the conjugate across the cellular membrane. Another example, PCT Publication No. WO 89/10134, discloses chimeric peptides which are adapted to deliver a neuropharmaceutical agent, conjugated with a transportable peptide, into the brain by transcytosis across the blood-brain barrier. In addition, oligonucleotides can be modified with hybridization-triggered cleavage agents (See, e.g., Krol et al., 1988, BioTech. 6:958 976) or intercalating agents (See, e.g., Zon, 1988, Pharm. Res. 5:539 549). To this end, the oligonucleotide can be conjugated to another molecule, e.g., a peptide, hybridization triggered cross-linking agent, transport agent, or hybridization-triggered cleavage agent.

The nucleic acid molecules can be inserted into vectors and used as gene therapy vectors. Gene therapy vectors can be delivered to a subject by, for example, intravenous injection, local administration (U.S. Pat. No. 5,328,470) or by stereotactic injection (See, e.g., Chen et al., 1994, Proc. Natl. Acad. Sci. 91:3054 3057). The pharmaceutical preparation of the gene therapy vector can include the gene therapy vector in an acceptable diluent, or can comprise a slow release matrix in which the vector is imbedded. Alternatively, where the vector can be produced intact from recombinant cells, e.g., retroviral vectors, the pharmaceutical preparation can include one or more cells producing the vector.

Any type of plasmid, cosmid, YAC or viral vector can be used to prepare the recombinant construct. Alternatively, vectors can be used which selectively target a tissue or cell type, e.g., viruses that infect bone cells. Further specificity can be realized by using a tissue-specific or cell-specific promoter in the expression vector.

In a specific embodiment, an expression vector is administered directly in vivo, where the vector is expressed to produce the encoded product. This can be accomplished by any of numerous methods known in the art, e.g., by placing a nucleic acid of the invention in an appropriate expression vector such that, upon administration, the vector becomes intracellular and expresses a nucleic acid of the invention. Such vectors can be internalized by using, for example, a defective or attenuated retroviral vector or other viral vectors that can infect mammalian cells (See e.g., U.S. Pat. No. 4,980,286).

Alternatively, an expression construct containing a nucleic acid of the invention can be injected directly into a target tissue as naked DNA. In another embodiment, an expression construct containing a nucleic acid of the invention can be introduced into a cell using microparticle bombardment, for example, by using a Biolistic gene gun (DuPont, Wilmington, Del.). In another embodiment, an expression construct containing a nucleic acid of the invention can be coated with lipids, or cell-surface receptors, or transfecting agents, such that encapsulation in liposomes, microparticles, or microcapsules facilitates access to target tissues and/or entry into target cells.

In yet another embodiment, an expression construct containing a nucleic acid of the invention is linked to a polypeptide that is internalized in a subset of cells or is targeted to a particular cellular compartment. In a further embodiment, the linked polypeptide is a nuclear targeting sequence that targets the vector to the cell nucleus. In another further embodiment, the linked polypeptide is a ligand that is internalized by receptor-mediated endocytosis in cells expressing the respective receptor for the ligand (See e.g., Wu and Wu, 1987, J. Biol. Chem. 262:4429 4432).

In another embodiment, nucleic acid-ligand complexes can be formed such that the ligand comprises a fusogenic viral peptide, which disrupts endosomes, thereby allowing the nucleic acid to avoid lysosomal degradation. In another embodiment, a nucleic acid of the invention can be targeted in vivo via a cell-specific receptor resulting in cell-specific uptake and expression (See e.g., International Patent Publications WO 92/06180, WO 92/22635, WO 92/20316, and WO 93/14188). For example, WO 92/06180 discloses that a virus or cell can be targeted to a target cell for internalization in vivo by introducing a receptor-specific molecule onto the surface of the virus or cell to produce a modified virus or cell which specifically binds to a receptor on the surface of the target cell, resulting in internalization by the target cell. Another example, WO 93/14188, discloses the use of a genetically engineered retroviral packaging cell line that has altered the viral envelope such that it contains a peptide that will bind to a molecule on the membrane of the target cell for the transfer of genetic information. Still other examples, WO 92/22635 and WO 92/20316, disclose a molecular complex for targeting a gene to a specific cell in vivo comprising an expressible gene complexed to a carrier that is a conjugate of a gene binding agent and a cell-specific binding agent, which is specific for a receptor that mediates internalization of bound ligands by endocytosis.

In yet another embodiment, a nucleic acid of the invention is introduced intracellularly and, by homologous recombination, can transiently or stably be incorporated within the host cell DNA, which then allows for its expression, (Koller and Smithies, 1989, Proc. Natl. Acad. Sci. 86:8932 8935; Zijlstra et al., 1989, Nature 342:435 438).

In one embodiment, viral vectors are used that contain nucleic acids encoding compounds that activate cytokine receptors (i.e., cytokines or antibodies), or compounds that activate molecules expressed on activated immune cells (See, e.g., Miller et al., 1993, Meth. Enzymol. 217:581 599). In a specific embodiment, a viral vector that contains nucleic acid sequences encoding 4-1BB ligand, or anti-4-1BB immunoglobulin, and/or IL-12 are used. For example, a retroviral vector can be used in which sequences not necessary for packaging of the viral genome and integration into host cell DNA have been deleted, and nucleic acid sequences encoding 4-1BB ligand, or anti-4-1BB immunoglobulin, or IL-12 are cloned into the vector, thereby facilitating delivery of the transgene into a subject. Greater detail about retroviral vectors is available in Boesen et al., 1994, Biotherapy 6:291 302, which describes the use of a retroviral vector to deliver the mdr1 gene to hematopoietic stem cells.

Other viral vectors can be used for gene therapy approaches in accordance with the invention. For example, adenoviruses are useful for delivering gene constructs to respiratory epithelia. Other targets for adenovirus-based delivery systems are the liver, the central nervous system, endothelial cells, and muscle cells. Moreover, adenoviruses are able to infect non-dividing cells (See, e.g., Rosenfeld et al., 1991, Science 252:431 434; Rosenfeld et al., 1992, Cell 68:143 155; Mastrangeli et al., 1993, J. Clin. Invest. 91:225 234; Kozarsky and Wilson, 1993, Curr. Opin. Genet. Develop. 3:499 503; Bout et al., 1994, Hum. Gene Ther. 5:3 10; PCT Publication No. WO 94/12649; and Wang et al., 1995, Gene Ther. 2:775 783).

Accordingly, adeno-associated virus can also be used in the gene therapy approaches of the present invention (See, e.g., Walsh et al., 1993, Proc. Soc. Exp. Biol. Med. 204:289 300; U.S. Pat. No. 5,436,146).

In this embodiment, the nucleic acid is introduced into a cell prior to administration in vivo of the resulting recombinant cell. Such introduction can be carried out by any method known in the art, including, but not limited to, transfection, electroporation, microinjection, infection with a viral or bacteriophage vector containing the nucleic acid sequences, cell fusion, chromosome-mediated gene transfer, microcell-mediated gene transfer, and spheroplast fusion. Numerous techniques are known in the art for the introduction of foreign genes into cells (See, e.g., Maniatis et al., 1989; Current Protocols, 2000; Loeffler and Behr, 1993, Meth. Enzymol. 217:599 618; Cohen et al., 1993, Meth. Enzymol. 217:618 644; Cline, 1985, Pharmacol. Ther. 29:69 92) and can be used in accordance with the present invention. In a preferred embodiment, the technique stably transfers a nucleic acid of the invention to a target cell, such that the nucleic acid is inherited by the cell's progeny.

The resulting recombinant cells can be delivered to a subject by various methods known in the art, and the skilled artisan would appreciate appropriate modes of administration. For example, intravenous administration may be the preferred mode of administration for recombinant hematopoietic stem cells. The number of recombinant cells to be administered to a subject can be determined by one skilled in the art, and would include a consideration of factors such as the desired effect, the disease state, and the mode of administration.

Cells into which a nucleic acid of the invention can be introduced for purposes of gene therapy include, but are not limited to, epithelial cells, endothelial cells, keratinocytes, fibroblasts, muscle cells, hepatocytes, blood cells (e.g., B lymphocytes, T lymphocytes, eosinophils, granulocytes, macrophages, megakaryocytes, monocytes, neutrophils), stem cells or progenitor cells (e.g., undifferentiated cells obtained from adipose, bone marrow, blood, fetal liver, and umbilical cord (See, e.g., Rheinwald, 1980, Meth. Cell Bio. 21A:229; International Publication No. WO 94/08598; Pittelkow and Scott, 1986, Mayo Clinic Proc. 61:771; and Stemple and Anderson, 1992, Cell 71:973 985). The cells used for introduction of a nucleic acid of the invention can be autologous or non-autologous. In a preferred embodiment, the cells used for gene therapy are autologous to the subject.

One skilled in the art will appreciate that many different promoters can be used to drive expression of a nucleic acid of the invention. In one embodiment, the promoter comprises hormone-sensitive elements. For example, a promoter containing an androgen-sensitive enhancer would be activated to a greater degree in androgen-producing cells or adjacent tissues. Such an expression construct may be beneficial for targeting tissues secreting abnormally high levels of androgen. In another embodiment, the promoter comprises elements of a fibroblast-specific promoter. In a further embodiment, the fibroblast-specific promoter comprises promoter elements from synovial fibroblasts. Alternatively, the promoter comprises elements of promoters that are activated in aggressive rheumatoid arthritis synovial fibroblasts. In a particular embodiment, the promoter comprises a portion of a proglucagon promoter. In a non-limiting example, a viral vector is used in which the viral promoter is replaced fully, or in part, with at least parts of a proglucagon promoter. Such an expression construct would more specifically be expressed in proglucagon-expressing cells.

Gene therapy approaches can also be used in accordance with the present invention to inhibit antagonists of GLP, particularly DPP-IV. For example, ribozyme and triple helix molecules can be used to target gene products of a GLP inhibitor, or of an aberrant GLP gene, resulting in a decrease in GLP inhibitor protein or aberrant GLP protein. Techniques for the production and use of antisense ribozyme and/or triple helix molecules are well known to those of skill in the art and can be designed with respect to the nucleotide sequence encoding the amino acid sequence of the target gene, also known in the art.

In another embodiment, mutations can be introduced into the gene encoding the GLP receptor resulting in an altered sequence that activates the receptor thus simulating increased GLP receptor binding (U.S. Pat. No. 6,077,949). The application of automated gene synthesis techniques provides an opportunity for generating sequence variants of the naturally occurring GLP receptor gene. The skilled artisan can appreciate that polynucleotides coding for variants of the GLP receptor can be generated by substitution of codons for those represented in the naturally occurring polynucleotide sequences provided herein. In addition, polynucleotides coding for synthetic variants of the GLP receptor herein provided can be generated which incorporate from 1 to 20, e.g., from 1 to 5, amino acid substitutions, or deletions or additions. The modified GLP receptor can be placed in an expression vector and administered to a subject in need of treatment to increase receptor activity in a desired tissue.

Antisense Therapy.

In one embodiment, an antisense approach to gene therapy can be used to treat an alcohol and drug dependency. Antisense approaches to gene therapy involve the use of riboprobes that can hybridize to a portion of the target mRNA. Additionally, non-ribose antisense constructs are contemplated in the present invention including, but not limited to, peptide nucleic acids (PNA), LNA, phosphine analogues, phosphotionates, and PEGA modified antisense constructs. Preventing transcription of a GLP antagonist will enhance GLP activity. The skilled artisan will recognize that absolute complementarity is not required, such that some degree of mismatch can result in, at least, transitory duplex formation. In one non-limiting example, the antisense riboprobe binds to the target mRNA transcript and prevents its translation. In one embodiment, the target mRNA encodes a GLP antagonist. In another embodiment, the target mRNA is an aberrant GLP mRNA.

Riboprobes that are complementary to the 5′ untranslated sequences, up to and including the AUG initiation codon, can be used effectively to inhibit translation of a GLP mRNA. Riboprobes complementary to the 3′ untranslated sequences of mRNAs also can be effective at inhibiting GLP mRNA translation (See, e.g., Wagner, 1994, Nature 372:333 335). Moreover, antisense riboprobes complementary to mRNA coding regions can be used in accordance with the invention.

Preferably, in vitro studies are performed to assess the ability of an antisense riboprobe to inhibit gene expression. These studies typically use controls which distinguish between antisense-mediated inhibition of gene expression and nonspecific biological effects of riboprobes. Preferably, these studies compare antisense-mediated changes in the levels of the target RNA or target protein with levels of an internal control RNA or protein.

In one embodiment, a recombinant DNA construct comprising an antisense riboprobe under the control of a pol III or pol II promoter is used to generate antisense riboprobes in a cell. The use of such a construct to transfect target cells in the subject can result in the transcription of sufficient amounts of a riboprobe to reduce or inhibit mRNA and/or protein expression. In one embodiment, the mRNA is a GLP inhibitor mRNA. In another embodiment, the mRNA is an aberrant GLP mRNA. Low transfection rates or low transcription activity of the DNA construct can nevertheless generate sufficient antisense molecules to demonstrate clinical effectiveness.

In another embodiment, a GLP inhibitor antisense nucleic acid sequence, or an aberrant GLP antisense nucleic acid sequence, is cloned into an expression vector, preferably a mammalian expression vector.

In another embodiment, aberrant GLP or GLP inhibitor antisense nucleic acid molecules of the invention are cloned into a vector, which is designed to target the vector (and thereby target expression of the antisense riboprobe) to specific tissues or cell-types. For example, an antisense riboprobe can be linked to peptides or antibodies that specifically bind receptors or antigens expressed on the target cell surface, thereby targeting the vector to the cells.

In another embodiment, the vector comprises a promoter that is more highly activated in diseased cells or tissues, as compared to normal cells or tissues.

Ribozyme Therapy.

Ribozyme therapy can be used to treat an alcohol and drug dependency.

Ribozymes are enzymatic RNA molecules capable of catalyzing the specific cleavage of a single-stranded nucleic acid, such as an mRNA (See, e.g., Rossi, 1994, Curr. Biol. 4:469 471). The mechanism of ribozyme action involves sequence-specific hybridization of the ribozyme molecule to complementary target RNA, followed by an endonucleolytic cleavage. The composition of ribozyme molecules include one or more sequences complementary to the target gene mRNA, and catalytic sequences responsible for mRNA cleavage (see e.g., U.S. Pat. No. 5,093,246 which is incorporated by reference in its entirety). Thus, ribozymes (e.g., hammerhead ribozymes) can be used to catalytically cleave mRNA transcripts thereby inhibiting the expression of a protein encoded by a particular mRNA (See, e.g., Haselhoff and Gerlach, 1988, Nature 334:585 591). A ribozyme having specificity for a nucleic acid molecule encoding a polypeptide of the invention can be designed based upon the nucleotide sequence of the nucleic acid molecules of the invention. Accordingly, in one embodiment, an engineered hammerhead motif ribozyme molecule specifically and efficiently catalyzes endonucleolytic cleavage of RNA sequences encoding a GLP antagonist of the invention.

In another embodiment, an mRNA encoding a polypeptide of the invention is used to select a catalytic RNA having a specific ribonuclease activity from a pool of RNA molecules (See, e.g., Bartel and Szostak, 1993, Science 261:1411 1418).

Specific ribozyme cleavage sites within a potential RNA target are identified by scanning the molecule of interest for ribozyme cleavage sites, which include the sequences GUA, GUU and GUC. Once identified, short RNA sequences of approximately 15 to 20 ribonucleotides corresponding to a cleavage site of a target gene are evaluated for predicted structural features, such as secondary structure, that may make the oligo-nucleotide suitable. The suitability of candidate sequences also can be evaluated by testing their ability to hybridize with complementary oligonucleotides, using for example, ribonuclease protection assays.

Triple-Helix Therapy.

In one embodiment, nucleic acid molecules that form triple helical structures are used to treat an alcohol and drug dependency. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells (See, e.g., Helene, 1991, Antican. Drug Des. 6:569 584; Helene, 1992, Ann. N.Y. Acad. Sci. 660:27 36; Maher, 1992, Bioassays 14:807 815).

Nucleic acid molecules to be used to inhibit transcription by triple helix formation can be single stranded oligonucleotides. The base composition of these oligonucleotides can be designed to promote triple helix formation via Hoogsteen base pairing rules, preferably with long stretches of purines or pyrimidines on one strand of the duplex. Nucleotide sequences can be pyrimidine-based thereby resulting in TAT and CGC+triplet across the three associated strands of the resulting triple helix. The pyrimidine-rich molecules provide base complementarity to a purine-rich region of a single strand of the duplex in a parallel orientation to that strand. Purine-rich nucleic acid molecules also can be chosen, for example, containing a stretch of guanine residues. These molecules can form a triple helix with a DNA duplex that is rich in GC pairs, in which most of the purine residues are located on a single strand of the targeted duplex, resulting in GGC triplets across the three strands in the triplex.

Additionally, the number of potential sequences that can be targeted for triple helix formation can be increased by creating a “switchback” nucleic acid molecule. Switchback molecules are synthesized in an alternating 5′-3′,3′-5′ manner, such that the molecule first hybridizes with one strand of a duplex, followed by hybridization with another strand, thus eliminating the requirement for a stretch of purines or pyrimidines on one strand of a duplex.

Ribozyme and triple helix molecules of the invention can be prepared by any method known in the art for the synthesis of DNA or RNA molecules (e.g., oligodeoxyribonucleotides or oligoribonucleotides). Such methods include, for example, solid phase phosphoramidite chemical synthesis.

These oligonucleotides can be administered directly, for example, via injection. Alternatively, RNA molecules can be generated in vitro or in vivo by transcription of DNA sequences. Such DNA sequences can be incorporated into a wide variety of vectors known in the art that feature a suitable RNA polymerase promoter such as, for example, a T7 or SP6 polymerase promoter. In a preferred embodiment, a bone-cell specific promoter is used to produce an expression vector comprising a nucleic acid sequence of the invention. In another preferred embodiment, a bone-specific promoter is used to produce an expression vector comprising a nucleic acid sequence of the invention.

Antibody Therapy.

The invention also encompasses the use of antibody therapy to treat an alcohol and drug dependency. In one embodiment, nucleic acid molecules comprising sequences encoding antibodies that bind to a GLP antagonist are administered via gene therapy. In a particular embodiment, recombinant cells are used that contain nucleic acid sequences encoding antibodies to GLP antagonist polypeptides of the invention. The gene construct is expressed such that the recombinant antibody is secreted or expressed on the cell surface. The recombinant cells are then administered in vivo for therapeutic effect.

GLP antibodies of the invention, including antibodies conjugated to therapeutic moieties, can be administered to an individual alone or in combination with an anti-osteoporosis agent, anti-obesity agent, growth factor or hormone. In one embodiment, an antibody directed to a GLP inhibitor polypeptide is administered first, followed by an anti-osteoporosis agent, anti-obesity agent, growth factor, or hormone within 24 hours. The treatment cycle can be repeated if warranted by the clinical response of the patient. Furthermore, the antibody, anti-osteoporosis agent, growth factor, or hormone can be administered via separate routes, such as for example, by intravenous and intramuscular administration.

Still another aspect of the invention is a pharmaceutical composition comprising an antibody of the invention and a pharmaceutically acceptable carrier. In preferred embodiments, the pharmaceutical composition contains an antibody of the invention, a GLP molecule, and a pharmaceutically acceptable carrier.

Vaccine Therapy.

Vaccine therapy can be used to treat an alcohol and drug dependency. Vaccine therapy can be administered to a subject in need of such treatment, e.g., a subject expressing an aberrant GLP variant or an aberrant intermediate in the GLP cascade. The nucleotides of the invention, including variants and derivatives, can be used as vaccines, e.g., by genetic immunization. Genetic immunization is particularly advantageous as it stimulates a cytotoxic T-cell response but does not utilize live attenuated vaccines, which can revert to a virulent form and infect the host causing the very infection sought to be prevented. As used herein, genetic immunization comprises inserting the nucleotides of the invention into a host, such that the nucleotides are taken up by cells of the host and the proteins encoded by the nucleotides are translated. These translated proteins are then either secreted or processed by the host cell for presentation to immune cells and an immune reaction is stimulated. Preferably, the immune reaction is a cytotoxic T cell response; however, a humeral response or macrophage stimulation is also useful in preventing future infections. The skilled artisan will appreciate that there are various methods for introducing foreign nucleotides into a host animal and subsequently into cells for genetic immunization, for example, by intramuscular injection of about 50 mg of plasmid DNA encoding the proteins of the invention solubilized in 50 ml of sterile saline solution, with a suitable adjuvant (See, e.g., Weiner and Kennedy, 1999, Sci. Am. 7:50 57; Lowrie et al., 1999, Nature 400:269 271).

Kits

The invention also encompasses kits for detecting the presence of a polypeptide or nucleic acid of the invention in a biological sample (a test sample). Such kits can be used to determine if a subject is suffering from or is at increased risk of developing a disorder associated with aberrant expression of a polypeptide of the invention as discussed, for example, in sections above relating to uses of the pharmaceutical compositions of the invention.

For example, kits can be used to determine if a subject is suffering from or is at increased risk of developing an alcohol and drug dependency.

In another example, kits can be used to determine if a subject is suffering from or is at risk for disorders that are associated with aberrant expression of a polypeptide of the invention.

The kit, for example, can comprise a labeled compound or agent capable of detecting the polypeptide or mRNA encoding the polypeptide in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for observing that the tested subject is suffering from or is at risk of developing a disorder associated with aberrant expression of the polypeptide if the amount of the polypeptide or mRNA encoding the polypeptide is above or below a normal level.

For antibody-based kits, the kit can comprise, for example: (1) a first antibody (e.g., attached to a solid support) which binds to a GLP polypeptide; and, optionally, (2) a second, different antibody which binds to either the polypeptide or the first antibody and is conjugated to a detectable agent.

For oligonucleotide-based kits, the kit can comprise, for example: (1) an oligonucleotide, e.g., a detectably labeled oligonucleotide, which hybridizes to a nucleic acid sequence encoding a polypeptide of the invention or (2) a pair of primers useful for amplifying a nucleic acid molecule encoding a polypeptide of the invention. The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of the polypeptide.

The invention provides a kit containing an antibody of the invention conjugated to a detectable substance, and instructions for use.

The pharmaceutical compositions of the invention can be included in a container, pack, or dispenser together with instructions for administration.

The GLP-1 like peptides can be made by solid state chemical peptide synthesis. GLP-1 can also be made by conventional recombinant techniques using standard procedures described in, for example, Sambrook and Maniaitis. “Recombinant”, as used herein, means that a protein is derived from recombinant (e.g., microbial or mammalian) expression systems which have been genetically modified to contain an expression gene for GLP-1 or its biologically active analogues.

The GLP-1 like peptides can be recovered and purified from recombinant cell cultures by methods including, but not limited to, ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. High performance liquid chromatography (HPLC) can be employed for final purification steps.

The polypeptides of the present invention may be a naturally purified product, or a product of chemical synthetic procedures, or produced by recombinant techniques from prokaryotic or eukaryotic hosts (for example by bacteria, yeast, higher plant, insect and mammalian cells in culture or in vivo). Depending on the host employed in a recombinant production procedure, the polypeptides of the present invention are generally non-glycosylated, but may be glycosylated.

GLP-1 activity can be determined by standard methods, in general, by receptor-binding activity screening procedures which involve providing appropriate cells that express the GLP-1 receptor on their surface, for example, insulinoma cell lines such as RINmSF cells or INS-1 cells. See also Mosjov, S. (1992) and EP0708170A2. In addition to measuring specific binding of tracer to membrane using radioimmunoassay methods, cAMP activity or glucose dependent insulin production can also be measured. In one method, a polynucleotide encoding the receptor of the present invention is employed to transfect cells to thereby express the GLP-1 receptor protein. Thus, for example, these methods may be employed for screening for a receptor agonist by contacting such cells with compounds to be screened and determining whether such compounds generate a signal, i.e. activate the receptor.

Polyclonal and monoclonal antibodies can be utilized to detect purify and identify GLP-1 like peptides for use in the methods described herein. Antibodies such as ABGA1178 detect intact unspliced GLP-1 (1-37) or N-terminally-truncated GLP-1 (7-37) or (7-36) amide. Other antibodies detect on the very end of the C-terminus of the precursor molecule, a procedure which allows by subtraction to calculate the amount of biologically active truncated peptide, i.e. GLP-1 (7-37) or (7-36) amide. (Orskov et al. Diabetes, 1993, 42:658-661; Orskov et al. J. Clin. Invest. 1991, 87:415-423).

Other screening techniques include the use of cells which express the GLP-1 receptor, for example, transfected CHO cells, in a system which measures extracellular pH or ionic changes caused by receptor activation. For example, potential agonists may be contacted with a cell which expresses the GLP-1 protein receptor and a second messenger response, e.g. signal transduction or ionic or pH changes, may be measured to determine whether the potential agonist is effective.

The GLP-1 derivative of the present invention can be combined with a pharmaceutically acceptable carrier, diluent, excipient or absorption promoter to prepare a pharmaceutical composition. The absorption promoter includes, for example, a chelating agent (for example, EDTA, citric acid, salicylate), a surfactant (for example, sodium dodecylsulfate (SDS)), a non-surfactant (for example, unsaturated cyclic urea) and bile acid salts (for example, sodium deoxycholate, sodium taurocholate). Such pharmaceutical compositions can be produced by methods known in the field of pharmaceutical manufacturing. These pharmaceutical preparations are suitable for administration via a mucous membrane in the nasal cavity or the like, and can be used alone or in combination with other therapeutic agents. The GLP-1 derivative of the present invention can also be formed into injections, oral preparations and the like other than the preparation for mucous membrane administration. The formulations should suit the mode of administration and are readily ascertained by those of skill in the art. The GLP-1 peptide may also be used in combination with agents known in the art that enhance the half-life in vivo of the peptide in order to enhance or prolong the biological activity of the peptide. For example, a molecule or chemical moiety may be covalently linked to the composition of the present invention before administration thereof. Alternatively, the enhancing agent may be administered concurrently with the composition. Still further, the agent may comprises a molecule that is known to inhibit the enzymatic degradation of GLP-1 like peptides may be administered concurrently with or after administration of the GLP-1 peptide composition. Such a molecule may be administered, for example, orally or by injection.

Using methods known in the technical field of the present invention, the composition of the present invention can be formed into a preparation to provide continuous or sustained release of the active ingredient immediately after administration into patients. For example, suitable macromolecules (for example, polyester, polyamino acid, polyvinyl pyrrolidone, ethylene vinyl acetate, methyl cellulose, carboxymethyl cellulose and protamine sulfate), or a polymer substance such as polyester, polyamino acid, hydrogel, poly(lactic acid) or ethyl vinyl acetate copolymer can be used to form a complex of the peptide of the present invention or to absorb the peptide of the present invention, in order to produce a preparation showing controlled release. Instead of mixing the peptide with particles of these polymers, the peptide of the present invention can be encapsulated in microcapsules produced by coacervation techniques or interfacial polymerization, microcapsules including hydroxymethyl cellulose or gelatin, in colloidal drug delivery system (for example, liposomes, albumin, microspheres, microemulsion, nano-particles and nano-capsules) or in microemulsion.

In the present invention, a preparation with further improvement in absorption of the peptide of the present invention via mucous membranes can be produced by absorbing the peptide of the present invention onto a charge-regulated fat emulsion prepared according to JP-A 8-27018. As the charge regulator, at least one substance selected from various acidic phospholipids and salts thereof, various fatty acids and salts thereof, bile acids and salts thereof is used. The acidic phospholipids and salts thereof include, but are not limited to, phosphatidyl serine, phosphatidyl glycerol, phosphatidyl inositol, phosphatidic acid and salts thereof. The fatty acids and salts thereof are not particularly limited either, but are preferably C6 or more fatty acids and salts thereof. The bile acids and salts thereof include, but are not limited to, dehydrocholic acid, deoxycholic acid, taurocholic acid and salts thereof. By selecting the charge regulator and establishing the concentration of the charge-regulated fatty emulsion, the pharmaceutical composition of the present invention suitable for administration site can be prepared.

The present invention encompasses the use of drugs or compounds or combinations of drugs or compounds to treat addictive and compulsive diseases and disorders, particular alcohol-related diseases and disorders. The present invention further encompasses the use of adjunctive treatments and therapy such as psychosocial management regimes, hypnosis, and acupuncture.

In some embodiments, a first compound and a second compound are administered nearly simultaneously. In other embodiments, a first compound is administered prior to the second compound. In yet other embodiments, the first compound is administered subsequent to the second compound. If three or more compounds are administered, one of ordinary skill in the art will appreciate that the three or more compounds can be administered simultaneously or in varying order.

In certain embodiments disclosed herein, an individual is given a pharmaceutical composition comprising a combination of two or more compounds to treat or prevent an addiction-related disease or disorder or impulse control-related disease or disorder. In some of these embodiments, each compound is a separate chemical entity. However, in other embodiments, the at least two compounds can be joined together by a chemical linkage, such as a covalent bond, so that the at least two different compounds form separate parts of the same molecule. In one aspect, the chemical linkage is selected such that after entry into the body, the linkage is broken, such as by enzymatic action, acid hydrolysis, base hydrolysis, or the like, and the two separate compounds are then formed.

With respect to alcohol-related disorders, including but not limited to alcohol abuse and alcohol dependence, at least two compounds selected from the group consisting of topiramate, ondansetron, and naltrexone, and analogs, derivatives, and modifications thereof, and pharmaceutically acceptable salts thereof, can be used to decrease ethanol consumption associated with such alcohol-related disorders. In one aspect, topiramate and ondansetron are used. Accordingly, the present invention provides a method for treating or preventing alcohol-related disorders based on ethanol consumption, comprising administering to a subject in need of such treatment or prevention an effective amount of at least two compounds selected from the group consisting of topiramate, ondansetron, and naltrexone, and analogs, derivatives, and modifications thereof or a pharmaceutically acceptable salt thereof. In a further aspect, the combination pharmacotherapy treatment is used in conjunction with behavioral modification or therapy.

In one embodiment the opioid antagonist is selected from the group consisting of an opioid antagonist selected from the group consisting of naltrexone (17-(cyclopropylmethyl)-4,5-epoxy-3,14-dihydroxy-5α-morphinan-6-one, ReVia, Trexan), nalmefene (17-(cyclopropylmethyl)-4,5-epoxy-6-methylene-5α-morphinan-3,14-dio-1, Revex) (also Nalmetrene, JF 1, Incystene, Arthene, Fenarc and Cervene), nalorphine (7,8-didehydro-4,5-epoxy-17-(2-propenyl)-morphinan-3,6-diol, Miromorfalil), naloxone (4,5-epoxy-3,14-dihydroxy-17-(2-propenyl)-morphinan-6-one, Narcan) (also naloxone hydrochloride), naltriben (17-(cyclopropylmethyl)-6,7-didehydro-3,14β-dihydroxy-4,5α-epoxy-6,7-2′,3′-benzo[b]furanomorphinan), naltrindole (17-(cyclopropylmethyl)-6,7-dehydro-4,5α-epoxy-3,14-dihydroxy-6,7-2-′,3′-indolomorphinan, NTI), cyprodime ((−)-N-(cyclopropylmethyl)-4,14-dimethoxy-morphinan-6-one), DPI-2505 ([3a,4(Z),5a]-4-[[4-(2-butenyl)-3,5-dimethyl-1-piperazinyl](3-hydroxyphen-yl)methyl]-N,N-diethylbenzamide monohydrochloride), and analogs, derivatives, and modifications thereof or a pharmaceutically acceptable salt thereof.

In one embodiment a serotonin receptor antagonist is selected from the group consisting of 1-(−)-cocaine, 2-bromo-CSD (BOL), 3-tropanyl-indole-3-carboxylate, 3-tropanyl-indole-3-carboxylate methiodide, amitriptine, carpipramine, chlorpromazine, cinanserin, clocapramine, clozapine, cyproheptadine, fluvoxamine, granisetron, imipramine, ketanserin, levomepromazine, LSD, LY-278,584, LY-53,857, MDL100907, MDL-11939, metergoline, methiothepin, methysergide, mianserin, milnacipran, mirtazapine, mosapramine, NAN-190, nortriptyne, olanzapine, paroxetine, perospirone, piperazine, p-NPPL, quetiapine, risperidone, ritanserin, sarpogrelate, SB-206553, SDZ-205,557, trazodone, and xylamidine

The present invention encompasses biologically active analogs, homologs, derivatives, and modifications of the compounds of the invention. Methods for the preparation of such compounds are known in the art.

The effectiveness of treatment or prevention of alcohol-related diseases and disorders can be detected and measured in several ways. For example, subjects can self-report according to guidelines and procedures set up for such reporting. Objective measures of alcohol consumption include the use of breath alcohol meter readings, measuring serum CDT levels, and measuring serum γ-glutamyl transferase (GGT) levels. Urinary 5-HTOL may also be measured and is an indicator of recent alcohol consumption. 5-HTOL is a minor metabolite of 5-HT. More than one of these types of assays may be performed to ensure accuracy. Other subjective and objective measures are also known. These measurements can be taken or performed at various times before, during, and after treatment.

Administration of an effective amount of compounds of the invention, or pharmaceutically acceptable salts thereof, whether alone or in combination with a secondary therapeutic agent, to a subject will detectably treat or prevent ethanol consumption in the subject. In exemplary embodiments, administration of compounds of the invention, or pharmaceutically acceptable salts thereof, whether alone or in combination with additional therapeutic agents, will yield a reduction in ethanol consumption by at least about 10%, 20%, 30%, 50% or greater, up to about 75-90%, or about 95% or greater. The present compositions can also be administered to a subject in combination with behavioral therapy or interaction.

Included within the scope of this invention are the various individual anomers, diastereomers and enantiomers as well as mixtures thereof. In addition, the compounds of this invention also include any pharmaceutically acceptable salts, for example: alkali metal salts, such as sodium and potassium; ammonium salts; monoalkylammonium salts; dialkylammonium salts; trialkylammonium salts; tetraalkylammonium salts; and tromethamine salts. Hydrates and other solvates of the compounds are included within the scope of this invention.

Additional therapeutic agents administered as combination therapies to treat alcohol-related disorders can include traditional anti-alcohol agents and/or other agents. Useful anti-alcohol agents in combinatorial formulations and coordinate treatment methods of the invention include, but are not limited to: disulfuram (Litten et al., Expert Opin Emerg. Drugs 10(2):323-43, 2005); naltrexone (Volpicelli et al., Arch. Gen. Psychiatry 49:876-880, 1992; O'Malley et al., Arch. Gen. Psychiatry 49(11):881-887, 1992); acamprosate (Campral) (Swift, N. Engl. J. Med. 340(19):1482-1490, 1999); ondansetron (Pettinati et al., Alcohol Clin. Exp. Res. 24(7):1041-1049, 2000; Stoltenberg, Scott, Clinical & Experimental Research 27(12):1853-1859, 2003); sertraline (Zoloft) (Pettinati et al., Alcohol Clin. Exp. Res. 24(7):1041-1049, 2000); tiapride (Shaw et al., Br. J. Psychiatry 150:164-8, 1987); gamma hydroxybutyrate (Alcover) (Poldrugo F. and Addolorato G., Alcohol Alcoholism 34(1), 15-24, 1999); galanthamine (Novel pharmacotherapies and patents for alcohol abuse and alcoholism 1998-2001, Expert Opinion on Therapeutic Patents, Vol. 11, No. 10, pages 1497-1521 (2001); U.S. Pat. No. 5,932,238); nalmefene (Revex) (Drobes et al., Alcohol Clin Exp Res., 28(9):1362-70 (2004); naloxone (Julius, D., and Renault, P., eds., Narcotic Antagonists: Naltrexone Progress Report, NIDA Research Monograph Series, Number 9. DHEW Publication No. (ADM) 76-387, Bethesda, Md.: National Institute on Drug Abuse, 1976; Jenab and Inturrisi, Molecular Brain Research 27:95-102, 1994); desoxypeganine (Doetkotte et al., Alcoholism: Clinical & Experimental Research, International Society for Biomedical Research on Alcoholism 12th World Congress on Biomedical Alcohol Research, Sep. 29-Oct. 2, 2004, Heidelburg/Mannheim, Germany, 28(8) Supplement:25A, 2004); benzodiazepines (Ntais et al., Benzodiazepines for alcohol withdrawal, Cochrane Database Syst. Rev. (3):CD005063, 2005; Mueller T I et al., Alcohol Clin. Exp. Res. 29(8):1411-8, 2005); neuroleptics such as laevomepromazine (Neurocil) and thioridazine (Melleril); piracetam; clonidine; carbamazepine; clomethiazole (Distraneurin); levetiracetam; quetiapine (Monnelly et al., J. Clin. Psychopharmacol. 24(5):532-5, 2004); risperidone; rimonabant; trazodone (Janiri et al., Alcohol 33(4):362-5, 1998); topiramate (Johnson B A et al., Lancet 361:1677-1685, 2003); aripiprazole (Beresford et al., J. Clin. Psychopharmacol. 25(4):363-6, 2005); and modafinil (Saletu et al., Prog. Neuropsychopharmacol. Biol. Psychiatry 14(2):195-214, 1990); amperozide, and modafinil.

The sulfamate derivatives of topiramate, or any of the other compounds of the invention and their derivatives, analogs or modifications thereof, may be used in conjunction with one or more other drug compounds and according to the methods of the present invention so long as the pharmaceutical agent has a use that is also effective in treating alcohol-related disorders. Those of ordinary skill in the art will be able to identify readily those pharmaceutical agents that have utility with the present invention. Those of ordinary skill in the art will recognize also numerous other compounds that fall within the categories and that are useful according to the invention for treating alcohol-related disorders. In one aspect, the anti-alcohol compounds of the invention are used in combination with drugs useful for other conditions.

The other therapeutic agent can be an anti-nicotine agent. Useful anti-nicotine agents include, but are not limited to, clonidine and bupropion. The other therapeutic agent can be an anti-opiate agent. Useful anti-opiate agents include, but are not limited to, methadone, clonidine, lofexidine, levomethadyl acetate HCl, naltrexone, and buprenorphine. The other therapeutic agent can be an anti-cocaine agent. Useful anti-cocaine agents include, but are not limited to, desipramine, amantadine, fluoxidine, and buprenorphine. The other therapeutic agent can be an appetite suppressant. Useful appetite suppressants include, but are not limited to, fenfluramine, phenylpropanolamine, and mazindol. The other therapeutic agent can be an anti-lysergic acid diethylamide (“anti-LSD”) agent. Useful anti-LSD agents include, but are not limited to, diazepam. The other therapeutic agent can be an anti-phencyclidine (“anti-PCP”) agent. Useful anti-PCP agents include, but are not limited to, haloperidol.

The other therapeutic agent can be an anti-depression agent. Useful anti-depression agents include, but are not limited to, amitriptyline, clomipramine, doxepine, imipramine, trimipramine, amoxapine, desipramine, maprotiline, nortriptyline, protripylinc, fluoxetine, fluvoxamine, paroxetine, sertraline, venlafaxine, bupropion, nefazodone, trazodone, phenelzine, tranylcypromine, selegiline, clonidine, gabapentin, and 2-pyridinyl[7-(pyridine-4-yl)pyrazolo[1,5-a]pyrimidin-3-yl]methanone compounds having at least one substituent on both the 2- and 4-pyridinyl rings. Useful classes of antidepressant agents include without limitation monoamine oxidase inhibitors, selective serotonin reuptake inhibitors, tricyclic antidepressants, tetracyclic antidepressants, norepinephrine uptake inhibitors, selective norepinephrine reuptake inhibitors, and serotonin and norepinephrine uptake inhibitors.

The other therapeutic agent can be an anxiolytic agent. Useful anxiolytic agents include, but are not limited to, benzodiazepines, such as alprazolam, chlordiazepoxide, clonazepam, clorazepate, diazepam, halazepam, lorazepam, oxazepam, and prazepam; non-benzodiazepine agents, such as buspirone; and tranquilizers, such as barbiturates.

The other therapeutic agent can be an antipsychotic drug. Useful antipsychotic drugs include, but are not limited to, phenothiazines, such as chlorpromazine, mesoridazine besylate, thioridazine, acetophenazine maleate, fluphenazine, perphenazine, and trifluoperazine; thioxanthenes, such as chlorprothixene, and thiothixene; and other heterocyclic compounds, such as clozapine, haloperidol, loxapine, molindonc, pimozide, and risperidone. Exemplary anti-psychotic drugs include chlorpromazine HCl, thioridazine HCl, fluphenazine HCl, thiothixene HCl, and molindone HCl.

The other therapeutic agent can be an anti-obesity drug. Useful anti-obesity drugs include, but are not limited, to beta-adrenergic receptor agonists, for example beta-3 receptor agonists such as, but not limited to, fenfluramine; dexfenfluramine; sibutramine; bupropion; fluoxetine; phentermine; amphetamine; methamphetamine; dextroamphetamine; benzphetamine; phendimetrazine; diethylpropion; mazindol; phenylpropanolamine; norepinephrine; serotonin reuptake inhibitors, such as sibutramine; and pancreatic lipase inhibitors, such as orlistat.

The pharmaceutical compositions of the anti-alcoholism agents of the present invention can be prepared as a “prodrug”, which is an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug, or may demonstrate increased palatability or be easier to formulate. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to provide the active moiety.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts.

One type of administration encompassed by the methods of the invention is parenteral administration, which includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, and intrasternal injection, and kidney dialytic infusion techniques

Pharmaceutical compositions that are useful in the methods of the invention may be prepared, packaged, or sold in formulations suitable for oral, rectal, vaginal, parenteral, topical, pulmonary, intranasal, inhalation, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject, or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Lactulose can also be used as a freely erodible filler and is useful when the compounds of the invention are prepared in capsule form.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

In one aspect, a preparation in the form of a syrup or elixir or for administration in the form of drops may comprise active ingredients together with a sweetener, which is preferably calorie-free, and which may further include methylparaben or propylparaben as antiseptics, a flavoring and a suitable color.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of a dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents including naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, and intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil in water or water in oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute about 50% to about 99.9% (w/w) of the composition, and the active ingredient may constitute about 0.1% to about 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to about 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as about 0.1% (w/w) and as much as about 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, comprise about 0.1% to about 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or atomized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1% to 1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

A pharmaceutical composition of the invention may be prepared, packaged, or sold in a formulation suitable for intramucosal administration. The present invention provides for intramucosal administration of compounds to allow passage or absorption of the compounds across mucosa. Such type of administration is useful for absorption orally (gingival, sublingual, buccal, etc.), rectally, vaginally, pulmonary, nasally, etc.

In some aspects, sublingual administration has an advantage for active ingredients which in some cases, when given orally, are subject to a substantial first pass metabolism and enzymatic degradation through the liver, resulting in rapid metabolization and a loss of therapeutic activity related to the activity of the liver enzymes that convert the molecule into inactive metabolites, or the activity of which is decreased because of this bioconversion.

In some cases, a sublingual route of administration is capable of producing a rapid onset of action due to the considerable permeability and vascularization of the buccal mucosa. Moreover, sublingual administration can also allow the administration of active ingredients which are not normally absorbed at the level of the stomach mucosa or digestive mucosa after oral administration, or alternatively which are partially or completely degraded in acidic medium after ingestion of, for example, a tablet.

Sublingual tablet preparation techniques known from the prior art are usually prepared by direct compression of a mixture of powders comprising the active ingredient and excipients for compression, such as diluents, binders, disintegrating agents and adjuvants. In an alternative method of preparation, the active ingredient and the compression excipients can be dry- or wet-granulated beforehand. In one aspect, the active ingredient is distributed throughout the mass of the tablet. WO 00/16750 describes a tablet for sublingual use that disintegrates rapidly and comprises an ordered mixture in which the active ingredient is in the form of microparticles which adhere to the surface of water-soluble particles that are substantially greater in size, constituting a support for the active microparticles, the composition also comprising a mucoadhesive agent. WO 00/57858 describes a tablet for sublingual use, comprising an active ingredient combined with an effervescent system intended to promote absorption, and also a pH-modifier.

The compounds of the invention can be prepared in a formulation or pharmaceutical composition appropriate for administration that allows or enhances absorption across mucosa. Mucosal absorption enhancers include, but are not limited to, a bile salt, fatty acid, surfactant, or alcohol. In specific embodiments, the permeation enhancer can be sodium cholate, sodium dodecyl sulphate, sodium deoxycholate, taurodeoxycholate, sodium glycocholate, dimethylsulfoxide or ethanol. In a further embodiment, a compound of the invention can be formulated with a mucosal penetration enhancer to facilitate delivery of the compound. The formulation can also be prepared with pH optimized for solubility, drug stability, and absorption through mucosa such as nasal mucosa, oral mucosa, vaginal mucosa, respiratory, and intestinal mucosa.

To further enhance mucosal delivery of pharmaceutical agents within the invention, formulations comprising the active agent may also contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10000 and preferably not more than 3000. Exemplary hydrophilic low molecular weight compounds include polyol compounds, such as oligo-, di- and monosaccharides such as sucrose, mannitol, lactose, L-arabinose, D-erythrose, D-ribose, D-xylose, D-mannose, D-galactose, lactulose, cellobiose, gentibiose, glycerin, and polyethylene glycol. Other examples of hydrophilic low molecular weight compounds useful as carriers within the invention include N-methylpyrrolidone, and alcohols (e.g., oligovinyl alcohol, ethanol, ethylene glycol, propylene glycol, etc.). These hydrophilic low molecular weight compounds can be used alone or in combination with one another or with other active or inactive components of the intranasal formulation.

When a controlled-release pharmaceutical preparation of the present invention further contains a hydrophilic base, many options are available for inclusion. Hydrophilic polymers such as a polyethylene glycol and polyvinyl pyrrolidone, sugar alcohols such as D-sorbitol and xylitol, saccharides such as sucrose, maltose, lactulose, D-fructose, dextran, and glucose, surfactants such as polyoxyethylene-hydrogenated castor oil, polyoxyethylene polyoxypropylene glycol, and polyoxyethylene sorbitan higher fatty acid esters, salts such as sodium chloride and magnesium chloride, organic acids such as citric acid and tartaric acid, amino acids such as glycine, beta-alanine, and lysine hydrochloride, and aminosaccharides such as meglumine are given as examples of the hydrophilic base. Polyethylene glycol, sucrose, and polyvinyl pyrrolidone are preferred and polyethylene glycol are further preferred. One or a combination of two or more hydrophilic bases can be used in the present invention.

The present invention contemplates pulmonary, nasal, or oral administration through an inhaler. In one embodiment, delivery from an inhaler can be a metered dose. An inhaler is a device for patient self-administration of at least one compound of the invention comprising a spray inhaler (e.g., a nasal, oral, or pulmonary spray inhaler) containing an aerosol spray formulation of at least one compound of the invention and a pharmaceutically acceptable dispersant. In one aspect, the device is metered to disperse an amount of the aerosol formulation by forming a spray that contains a dose of at least one compound of the invention effective to treat a disease or disorder encompassed by the invention. The dispersant may be a surfactant, such as, but not limited to, polyoxyethylene fatty acid esters, polyoxyethylene fatty acid alcohols, and polyoxyethylene sorbitan fatty acid esters. Phospholipid-based surfactants also may be used.

In other embodiments, the aerosol formulation is provided as a dry powder aerosol formulation in which a compound of the invention is present as a finely divided powder. The dry powder formulation can further comprise a bulking agent, such as, but not limited to, lactose, sorbitol, sucrose, and mannitol. In another specific embodiment, the aerosol formulation is a liquid aerosol formulation further comprising a pharmaceutically acceptable diluent, such as, but not limited to, sterile water, saline, buffered saline and dextrose solution. In further embodiments, the aerosol formulation further comprises at least one additional compound of the invention in a concentration such that the metered amount of the aerosol formulation dispersed by the device contains a dose of the additional compound in a metered amount that is effective to ameliorate the symptoms of disease or disorder disclosed herein when used in combination with at least a first or second compound of the invention.

Thus, the invention provides a self administration method for outpatient treatment of an addiction related disease or disorder such as an alcohol-related disease or disorder. Such administration may be used in a hospital, in a medical office, or outside a hospital or medical office by non-medical personnel for self administration.

Compounds of the invention will be prepared in a formulation or pharmaceutical composition appropriate for nasal administration. In a further embodiment, the compounds of the invention can be formulated with a mucosal penetration enhancer to facilitate delivery of the drug. The formulation can also be prepared with pH optimized for solubility, drug stability, absorption through nasal mucosa, and other considerations.

Capsules, blisters, and cartridges for use in an inhaler or insufflator may be formulated to contain a powder mix of the pharmaceutical compositions provided herein; a suitable powder base, such as lactose or starch; and a performance modifier, such as 1-leucine, mannitol, or magnesium stearate. The lactose may be anhydrous or in the form of the monohydrate. Other suitable excipients include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

For administration by inhalation, the compounds for use according to the methods of the invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of e.g., gelatin for use in an inhaler or insufflator may be formulated containing a powder mix of the drugs and a suitable powder base such as lactose or starch.

Typically, dosages of the compounds of the invention which may be administered to an animal, preferably a human, range in amount from about 1.0 μg to about 100 g per kilogram of body weight of the animal. The precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

In one embodiment the method for treating or preventing an addictive disease or disorder comprising administering a GLP activator in an amount of from 0.1 μg to 5000 μg per single dose, preferably 1 μg to 2000 μg per single dose or more preferably 5 μg to 600 μg.

In another embodiment the treatment is by administering a GMP activator by subcutaneous or intramuscular injection in an amount to provide a dose of from 0.01 μg to 2000 μg of active ingredient per injection and not more than 2500 μg daily.

In another embodiment the treatment is by administering an effective amount of at least one GLP agent to the subject within the range of about 0.001 mg/kg to about 100 μg/kg.

In another embodiment the treatment is by administering an effective amount of the at least one protective agent to the subject within the range of about 0.01 mg/kg to about 10 mg/kg.

In another embodiment the treatment is by administering an effective amount of the at least one protective agent to the subject within the range of about 0.1 mg/kg to about 1 mg/kg.

In another embodiment the treatment is by administering a GMP activator in an amount of from 0.015 to 0.5 g per single dose and 0.80 g daily.

In another embodiment the treatment is by administering a GMP activator by subcutaneous or intramuscular injection in an amount to provide a dose of from 0.015 to 0.045 g of active ingredient per injection and not more than 0.120 gram daily.

In another embodiment the treatment is by administering an effective amount of at least one GLP agent administered to the subject within the range of about 0.001 mg/kg to about 100 mg/kg.

In another embodiment the treatment is by administering an effective amount of at least one protective agent administered to the subject within the range of about 0.01 mg/kg to about 10 mg/kg.

In another embodiment the treatment is by administering an effective amount of at least one protective agent to the subject within the range of about 0.1 mg/kg to about 1 mg/kg.

The compounds may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

In one embodiment, the invention also includes a kit comprising the compounds of the invention and an instructional material that describes administration of the compounds. In another embodiment, this kit comprises a (preferably sterile) solvent suitable for dissolving or suspending the composition of the invention prior to administering the compound to the mammal.

As used herein, an “instructional material” includes a publication, a recording, a diagram, or any other medium of expression that can be used to communicate the usefulness of the compounds of the invention in the kit for effecting alleviation of the various diseases or disorders recited herein. Optionally, or alternately, the instructional material may describe one or more methods of alleviating the diseases or disorders. The instructional material of the kit of the invention may, for example, be affixed to a container that contains a compound of the invention or be shipped together with a container that contains the compounds. Alternatively, the instructional material may be shipped separately from the container with the intention that the instructional material and the compound be used cooperatively by the recipient.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention, and are not to be construed as limiting in any way the remainder of the disclosure.

What is claimed is:

1. A method for treating or preventing an addictive disease or disorder comprising administering to a patient in need thereof an effective amount of a GLP activator; together with a pharmaceutically acceptable excipient or carrier for a time sufficient and under conditions effective to decrease alcohol dependency in the subject.

2. The method of claim 1, wherein the GLP activator is a compound selected from the group consisting of glucagon-like peptide-1 (GLP-1), GLP-1 analogs capable of binding and activating a GLP-1 receptor, agonist of the GLP-1 receptor, and pharmaceutically acceptable salts, esters or amide of any of the foregoing.

3. The method of claim 2, wherein the addictive disease or disorder is selected from the group consisting of alcohol-related diseases and disorders, obesity-related diseases and disorders, eating disorders, impulse control disorders, nicotine-related disorders, amphetamine-related disorders, methamphetamine-related disorders, cannabis-related disorders, cocaine-related disorders, hallucinogen use disorders, inhalant-related disorders, benzodiazepine abuse or dependence related disorders, and opioid-related disorders.

4. The method of claim 3, wherein the addictive disease or disorder is an alcohol-related disease or disorder.

5. The method of claim 4, wherein the alcohol-related disease or disorder is selected from the group consisting of early onset alcoholic, late onset alcoholic, alcohol-induced psychotic disorder with delusions, alcohol abuse, heavy drinking, excessive drinking, alcohol intoxication, alcohol withdrawal, alcohol intoxication delirium, alcohol withdrawal delirium, alcohol-induced persisting dementia, alcohol-induced persisting amnestic disorder, alcohol dependence, alcohol-induced psychotic disorder with hallucinations, alcohol-induced mood disorder, alcohol-induced or associated bipolar disorder, alcohol-induced or associated post traumatic stress disorder, alcohol-induced anxiety disorder, alcohol-induced sexual dysfunction, alcohol-induced sleep disorder, alcohol-induced or associated gambling disorder, alcohol-induced or associated sexual disorder, alcohol-related disorder not otherwise specified, alcohol intoxication, and alcohol withdrawal.

6. The method of claim 5, wherein the treatment reduces the frequency of alcohol consumption compared with the frequency before the treatment or compared with a control subject not receiving the treatment.

7. The method of claim 6, wherein the alcohol consumption comprises heavy drinking or excessive drinking.

8. The method of claim 5, wherein the treatment reduces the quantity of alcohol consumed compared with the amount of alcohol consumed before the treatment or compared with a control subject not receiving the treatment.

9. The method of claim 8, wherein the alcohol consumption comprises heavy drinking or excessive drinking.

10. The method of claim 5, wherein the treatment increases the abstinence rate of the subject compared with a control subject not receiving the treatment.

11. The method of claim 5, wherein the treatment reduces the average level of alcohol consumption compared with the level before the treatment or compared with a control subject not receiving the treatment.

12. The method of claim 3, wherein the treatment reduces alcohol consumption and increases abstinence compared with the alcohol consumption and abstinence before the treatment or compared with a control subject not receiving the treatment.

13. The method of claim 5, wherein the subject comprises a predisposition to early-onset alcoholism or late-onset alcoholism.

14. The method according to claim 1, wherein the administered compound is a GLP-1 (glucagon-like peptide-1) receptor agonist.

15. The method according to claim 14, wherein the GLP-1 receptor agonist is selected from the group consisting of Byetta (exenatide), Victoza (liraglutide), CJC-1131(a GLP-1-albumin drug affinity complex; DAC), ZP10 (an exendin-4 derivative; AVE-0010), BIM51077 (a human GLP-1 derivative; Taspoglutide), LY315902 (a DPP-IV-resistant GLP-1 analogue), LY307161 SR (a sustained release formulation of a GLP-1 analog), LY2199265 (an Fc immunoglobulin fusion protein), LY2428757 (a pegylated GLP-1 molecule) and NN9535 (a human GLP-1R agonist).

16. The method according to claim 1, wherein the administered compound is a DPP-4 inhibitor.

17. The method according to claim 14, wherein the DPP-4 inhibitor selected from the group consisting of sitagliptin, vildagliptin, saxagliptin, linagliptin, dutogliptin, gemigliptin, alogliptin, and berberine.

18. The method of claim 1 wherein the pharmaceutical carrier is selected from the group consisting of saline, buffered saline, dextrose, water, glycerol, ethanol, lactose, phosphate, mannitol, arginine, treholose, and combinations mixtures thereof.

19. The method of claim 1 wherein the administration is by a method selected from the group consisting of oral, intravenous infusion, subcutaneous injection, intramuscular injection, topical, depo injection, implantation, time-release mode, controlled-release mode, intracavitary, intranasal, inhalation, intratumor, intraocular intraperitoneal, intraorbital, intracapsular, intraspinal, intrasternal, intra-arterial; intradermal parenteral, transmucosal, nasal, rectal, intravaginal, sublingual, submucosal, transdermal, or transdermal patch route.

20. The method of claim 1 further comprising concurrent administration of an therapeutically effective amount of at least one compound, or biologically active analog, derivative, modification, or pharmaceutically acceptable salt thereof, selected from the group consisting of serotonergic agents, serotonin antagonists, selective serotonin re-uptake inhibitors, serotonin receptor antagonists, opioid antagonists, dopaminergic agents, dopamine release inhibitors, dopamine antagonists, norepinephrine antagonists, γ-amino-butyric acid agonists, γ-amino-butyric acid inhibitors, γ-amino-butyric acid receptor antagonists, γ-amino-butyric acid channel antagonists, glutamate agonists, glutamate antagonists, glutamine agonists, glutamine antagonists, anti-convulsant agents, N-methyl-D-aspartatc-blocking agents, calcium channel antagonists, carbonic anhydrase inhibitors, neurokinins, small molecules, peptides, vitamins, co-factors, and Corticosteroid Releasing Factor antagonists, thereby treating or preventing an addictive disease or disorder in a subject.

21. The method of claim 20 wherein the at least one compound is selected from the group consisting of topiramate, an opioid antagonist and a serotonin receptor antagonist, and pharmaceutically-acceptable salts thereof.

22. The method of claim 21 wherein the opioid antagonist is selected from the group consisting of an opioid antagonist selected from the group consisting of naltrexone (17-(cyclopropylmethyl)-4,5-epoxy-3,14-dihydroxy-5α-morphinan-6-one, ReVia, Trexan), nalmefene (17-(cyclopropylmethyl)-4,5-epoxy-6-methylene-5α-morphinan-3,14-dio-1, Revex) (also Nalmetrene, JF 1, Incystene, Arthene, Fenarc and Cervene), nalorphine (7,8-didehydro-4,5-epoxy-17-(2-propenyl)-morphinan-3,6-diol, Miromorfalil), naloxone (4,5-epoxy-3,14-dihydroxy-17-(2-propenyl)-morphinan-6-one, Narcan) (also naloxone hydrochloride), naltriben (17-(cyclopropylmethyl)-6,7-didehydro-3,14β-dihydroxy-4,5α-epo-xy-6,7-2′,3′-benzo[b]furanomorphinan), naltrindole (17-(cyclopropylmethyl)-6,7-dehydro-4,5α-epoxy-3,14-dihydroxy-6,7-2-′,3′-indolomorphinan, NTI), cyprodime ((−)-N-(cyclopropylmethyl)-4,14-dimethoxy-morphinan-6-one), DPI-2505 ([3a,4(Z),5a]-4-[[4-(2-butenyl)-3,5-dimethyl-1-piperazinyl](3-hydroxyphen-yl)methyl]-N,N-diethylbenzamide monohydrochloride), and pharmaceutically acceptable salt thereof.

23. The method of claim 21 wherein the serotonin receptor antagonist is selected from the group consisting of 1-(−)-cocaine, 2-bromo-CSD (BOL), 3-tropanyl-indole-3-carboxylate, 3-tropanyl-indole-3-carboxylate methiodide, amitriptine, carpipramine, chlorpromazine, cinanserin, clocapramine, clozapine, cyproheptadine, fluvoxamine, granisetron, imipramine, ketanserin, levomepromazine, LSD, LY-278,584, LY-53,857, MDL100907, MDL-11939, metergoline, methiothepin, methysergide, mianserin, milnacipran, mirtazapine, mosapramine, NAN-190, nortriptyne, olanzapine, paroxetine, perospirone, piperazine, p-NPPL, quetiapine, risperidone, ritanserin, sarpogrelate, SB-206553, SDZ-205,557, trazodone, and xylamidine.

24. The method according to claim 1, wherein the treatment is by administering the composition in an amount of from 0.1 μg to 5000 μg per single dose.

25. The method according to claim 1, wherein the treatment is by administering the composition by subcutaneous or intramuscular injection in an amount to provide a dose of from 0.01 μg to 2000 μg of active ingredient per injection and not more than 2500 μg daily.

26. The method of claim 2, wherein the effective amount of the at least one GLP agent administered to the subject is within the range of about 0.001 mg/kg to about 100 μg/kg.

27. The method of claim 5, wherein the effective amount of the at least one protective agent administered to the subject is within the range of about 0.01 mg/kg to about 10 mg/kg.

28. The method of claim 5, wherein the effective amount of the at least one protective agent administered to the subject is within the range of about 0.1 mg/kg to about 1 mg/kg.

29. The method according to claim 1, wherein the treatment is by administering the composition in an amount of from 0.015 to 0.5 g per single dose and not more than 0.80 g daily.

30. The method according to claim 1, wherein the treatment is by administering the composition by subcutaneous or intramuscular injection in an amount to provide a dose of from 0.015 to 0.045 g of active ingredient per injection and not more than 0.120 gram daily.

31. The method of claim 2, wherein the effective amount of the at least one GLP agent administered to the subject is within the range of about 0.001 mg/kg to about 100 mg/kg.

32. The method of claim 5, wherein the effective amount of the at least one protective agent administered to the subject is within the range of about 0.01 mg/kg to about 10 mg/kg.

33. The method of claim 5, wherein the effective amount of the at least one protective agent administered to the subject is within the range of about 0.1 mg/kg to about 1 mg/kg.

34. In one embodiment, the peripheral serotonin receptor antagonist is administered in an amount of at least about 0.01 mg per 100 kg body weight.


SEQUENCE LISTING (31 sequences)

SEQ ID NO: 1 - protein, 37 aa, Artificial Sequence
  Mammalian
HDEFERHAEGTFTSDVSSYLEGQAAKEFIAWLVKGRG

SEQ ID NO: 2 - protein, 36 aa, Artificial Sequence
  Mammalian
HDEFERHAEGTFTSDVSSYLEGQAAKEFIAWLVKGR

SEQ ID NO: 3 - protein, 31 aa, Artificial Sequence
  Mammalian
HAEGTFTSDVSSYLEGQAAKEFIAWLVKGRG

SEQ ID NO: 4 - protein, 30 aa, Artificial Sequence
  Mammalian
HAEGTFTSDVSSYLEGQAAKEFIAWLVKGR

SEQ ID NO: 5 - protein, 29 aa, Artificial Sequence
  Mammalian
EGTFTSDVSSYLEGQAAKEFIAWLVKGRG

SEQ ID NO: 6 - protein, 28 aa, Artificial Sequence
  Mammalian
EGTFTSDVSSYLEGQAAKEFIAWLVKGR

SEQ ID NO: 7 - protein, 39 aa, Artificial Sequence
  Heloderma Suspectum
HSDGTFTSDLSKQMEEEAVRLFIEWLKNGGPSSGAPPPS

SEQ ID NO: 8 - protein, 31 aa, Artificial Sequence
  Heloderma Suspectum
DLSKQMEEEAVRLFIEWLKNGGPSSGAPPPS

SEQ ID NO: 9 - protein, 39 aa, Artificial Sequence
  Heloderma Suspectum
HGEGTFTSDLSKQMEEEAVRLFIGWLKNGGPSSGAPPPS

SEQ ID NO: 10 - protein, 38 aa, Artificial Sequence
  Heloderma Suspectum
HSDATFTAEYSKLLAKLALQKYLESILGSSTSPRPPSS

SEQ ID NO: 11 - protein, 37 aa, Artificial Sequence
  Heloderma Suspectum
HSDATFTAEYSKLLAKLALQKYLESILGSSTSPRPPS

SEQ ID NO: 12 - protein, 35 aa, Artificial Sequence
  Heloderma Suspectum
HSDAIFTEEYSKLLAKLALQKYLASILGSRTSPPP

SEQ ID NO: 13 - protein, 35 aa, Artificial Sequence
  Heloderma Suspectum
HSDAIFTQQYSKLLAKLALQKYLASILGSRTSPPP

SEQ ID NO: 14 - protein, 30 aa, Artificial Sequence
  Description of Artificial Sequence Synthetic modified GLP-1 peptide
HXEGTFTSDVSSYLEGQAAKEFIAWLVKXR

SEQ ID NO: 15 - protein, 30 aa, Artificial Sequence; identical to SEQ ID NO: 14
  Description of Artificial Sequence Synthetic modified GLP-1 peptide

SEQ ID NO: 16 - protein, 37 aa, Artificial Sequence
  mamalian
XXHADGSFSDEMNTXLDXLAXRDFINWLXXTKITDXX

SEQ ID NO: 17 - protein, 30 aa, Artificial Sequence
  Description of Artificial Sequence Synthetic modified GLP-1 peptide
HAQGTFTSDVSSYLEGQAAKEFIAWLVKGR

SEQ ID NO: 18 - protein, 30 aa, Artificial Sequence; identical to SEQ ID NO: 17
  Description of Artificial Sequence Synthetic modified GLP-1 peptide

SEQ ID NO: 19 - protein, 30 aa, Artificial Sequence
  Description of Artificial Sequence Synthetic modified GLP-1 peptide
HAKGTFTSDVSSYLEGQAAKEFIAWLVKGR

SEQ ID NO: 20 - protein, 30 aa, Artificial Sequence
  Description of Artificial Sequence Synthetic modified GLP-1 peptide
HAEGTFTSDTSKYLEGQAAKEFIAWLVKGR

SEQ ID NO: 21 - protein, 30 aa, Artificial Sequence
  Description of Artificial SequenceSynthetic modified GLP-1 peptide
HAEGTFTSDVSKYLEGQAAKEFIAWLVKGR

SEQ ID NO: 22 - protein, 28 aa, Artificial Sequence
  Description of Artificial SequenceSynthetic modified GLP-1 peptide
HAQGTFTSDVSSYLQGEAAKQFIAWLVK

SEQ ID NO: 23 - protein, 29 aa, Artificial Sequence
  Description of Artificial Sequencemodified GLP-1 peptide
HAQGTFTSDVSSYLQGEAAKQFIAWLVKG

SEQ ID NO: 24 - protein, 30 aa, Artificial Sequence
  Description of Artificial SequenceSynthetic modified GLP-1 peptide
HAQGTFTSDVSSYLQGEAAKQFIAWLVKGR

SEQ ID NO: 25 - protein, 31 aa, Artificial Sequence
  Description of Artificial SequenceSynthetic modified GLP-1 peptide
HAQGTFTSDVSSYLQGEAAKQFIAWLVKGRG

SEQ ID NO: 26 - protein, 34 aa, Artificial Sequence
  Description of Artificial SequenceSynthetic modified GLP-1 peptide
HAQGTFTSDVSSYLQGEAAKQFIAWLVKGRGXXX

SEQ ID NO: 27 - protein, 31 aa, Artificial Sequence
  Description of Artificial SequenceSynthetic modified GLP-1 peptide
HAQGTFTSDVSSYLQGEAAXQFIAWLVXGXG

SEQ ID NO: 28 - protein, 31 aa, Artificial Sequence
  Description of Artificial SequenceSynthetic modified GLP-1 peptide
HAQGTFTSDVSSYLQGEAAKQFIAXLVKGRG

SEQ ID NO: 29 - protein, 31 aa, Artificial Sequence
  Description of Artificial SequenceSynthetic modified GLP-1 peptide
HAQGTFTSDYSKYLDSRRAKEFIAWLVKGRG

SEQ ID NO: 30 - protein, 31 aa, Artificial Sequence
  Description of Artificial SequenceSynthetic modified GLP-1 peptide
HXXXTFTSXVSSYLQGEAAKQFIAWLVKGRG

SEQ ID NO: 31 - protein, 31 aa, Artificial Sequence
  Description of Artificial SequenceFormula Sequence
XAQGTFTSDVSSYLQGEAAKQFIAWLVKGRG